(19) United States
(12) Patent Application Publication
(10) Pub. No.: US 2003/0077596 A1
(43) Pub. Date: Apr. 24, 2003

(54) Analytic method and reagent for use thereof

(76) Inventor: Erling Sundrehagen, Oslo (NO)

Correspondence Address: MERCHANT & GOULD PC, P.O. BOX 2903, MINNEAPOLIS, MN, 55402-0903, US

(21) Appl. No.: 10/019,866

(22) PCT Filed: Nov. 30, 2001
(86) PCT No.: PCT/NO01/00480

(30) Foreign Application Priority Data
Dec. 1, 2000 (NO) 20006130

(51) Int. Cl. C12Q 1/68; G01N 33/567; G06F 19/00; G01N 33/48; G01N 33/50

(52) U.S. Cl. 435/6; 435/7.21; 702/19

(57) ABSTRACT

The present invention relates to a method for determination of one or more analytes in a test sample or an aliquot of a test sample, as well as a reagent for use in the metod. The reagent according to the present invention comprises at least one type of specific binding molecule for each analyte that is to be quantitated, as well as fluorescent substances whose signals change as a result of admixing a sample to the magnet. Furthermore, the signal change may be used to calculate the concentration or concentrations of analytes, without separating different states of aggregation.

The present invention relates to a method for determination of one or more analytes in a test sample or an aliquot of a test sample and a reagent for use in the metod.

### BACKGROUND OF THE INVENTION

<<Analyte>> or <<analytes>> is the generic name of those substances for which qualitative or quantitative analysis is desired in a test sample material. An analyte is usually a well-defined molecule, but may be a collection of molecules resembling each other or carrying out the same function. Particulate materials, e.g. clusters of fatty proteins or classes of blood cells can also be referred to as analytes.

Quantitative chemical analysis of an analyte in a test material is also often referred to as determination of concentration. Concentration determination may also be carried out using a qualitative chemical analysis, whereby concentration determination provides information on whether the concentration of the analyte in the test material is higher than or lower than a given value.

The development of methods for chemical concentration determination of analytes in very low concentration in complex materials increased very rapidly after R. S. Yalow, S. A. Berson and associates developed what was later to be known a the immunochemical measurement methods. They made use of the principle that individuals of vertebrates create antibodies, most often in the form of immunoglobulins, as a specific immunological response to exposure to materials that the given animal's immune system perceives as alien to the animal. The classical and decisive work to reach this perception was published by S. A. Berson, R. S. Yalow, A. Bauman, M. A. Rothschild, and K. Newerly in the article “Insulin-[13] Metabolism in Human Subjects; Demonstration of Insulin Binding Globulin in the Circulation of Insulin-Treated Subjects” in J. Clin. Invest. 35 (1956): 170-190. By using these globulins, later referred to as antibodies, either in a purified form or in a mixture with other substances, e.g. in the form of blood serum, methods for specific identification or quantitation of a large number of analytes were developed. For many of these analytes there had previously not existed practically applicable measurement methods. Thins specific measurement methods for a range of blood proteins and proteins from other body fluids and tissue were developed, and later on for non-proteinaceous substances as well. Hormone analyses were developed at an early stage. The general method was named <<immunoassay>>, based on the reagent making the basis for the specificity of the analysis being the result of an immune response. This is well described in a large number of textbooks on the subject.

The test material undergoing quantitative analysis using those reagents can be described as complex biological materials. Typical test materials are blood, blood serum plasma, urine, feces, feces extracts and cerebrospinal fluid. The analytes usually constitute a very small part of the test material.

Test material that is to undergo chemical analysis may be in different states of aggregation, e.g. gaseous state, fluid state, solid state or mixtures of different states of aggregation. The reagents that are mixed with the test material to perform the quantitative analysis or analyses, may correspondingly be present in different states of aggregation, but typically consist of solutions or solid substances and/or combinations of solid substances and fluids. When the reagents are mixed with the test material, most often (but not always) a solution of substances in fluid will be formed. This solution is often referred to using the generic name <<the assay solution>>, and comprises both test material and those chemicals necessary for the quantitative analysis to take place.

In the early days after the invention of the immunossays, the source used for admixture of antibodies was antiserum, which is blood serum from the immunized animal, which also included other serum proteins, and where the antibodies constitute a smaller portion of the aggregate serum proteins. To this day a substantial amount of antibodies in the form of antiserum are sold, see e.g. catalogues from suppliers such as Chemicon Inc., California. However, early on the routine use of antibodies in the form of purified immunoglobulin classes was introduced, most commonly immunoglobulin type G, which is the most commonly used antibody reagent today.

The production of antibodies occurring when animals are used to alien substances (called antigens), often using active, targeted exposure in the form of so called vaccination or immunization, is characterized by a whole range of different cells in the body producing antibodies with different structures, but they have the common characteristic that they bind to (have affinity for) the antigen in question. These different antibodies, binding to the same antigen, have different strength and rates of binding, however, and are difficult to mate with constant qualities when producing antibodies over a long time span and using many animals.

It was therefore a considerable progress when Kölher and Milstein in 1974 developed the method for creating monoclonal antibodies, which made possible standardized antibodies with a reproducible chemical structure and homogeneous constant binding qualities. It is also known in the prior art how to fragment the antibodies and use those fragments of the antibodies that have binding qualities, instead of using the entire antibodies.

In the years following the invention of the monoclonal antibodies, using modern biotechnology, new methods were developed for producing binding proteins and fragments of proteins (also called peptides) with the desired binding qualities. A substantial step forward was made with the development of the phage display method, in which virus technology was used for production, exposure and choice of peptide structures for the production of reagents with specific binding characteristics.

The phage display technique was developed further when the gene sequence for parts of the antibody producing cells was incorporated and systematically varied or permuted in the phage display particles, as described by Collins J. and Röttgen P. (1994); “Hypervariable phagemid display gene banks for the selection of strongly binding ligands, including their use for the isolation of serine protease inhibitors”; European patent application 1994 000 108 689 (April 1994) taken further as U.S. Pat. No. 592,559 <<Phagemids and process of preparation>> issued Jul. 20, 1999, and by Collins, J., Röttgen, (1997); <<Cosmix-plexing a method for recombination . . . >> EP 97 101 539.1 (Jun. 2, 1997), filed by Cosmix GmbH PCT/EP98 9953 (Feb. 2, 1998) and WO 98 33901 (Jun. 8, 1998).

Further on, one started to use nucleic acid sequences (aptamers) as such as specific binding molecules in an immunoassay-like way, even though terminologically it is probably not quite correct to call these immunoassays. U.S. Pat. No. 5,567,588 <<Systematic evolution of ligands by exponential enrichment: Solution SELEX>> describes this aptamer technology. Finally, things went completely beyond the scope of the biological framework surrounding these binding assays when one started, synthetically, to produce large libraries of molecules, using systematic combination of smaller elements, so called combinatory libraries. These elements may partially or totally consist of amino acids in chains (called peptides), but could also consist of other building blocks.

So today there exists a very complex biological and synthetic collection of methods for creation of binding molecules of a higher or lower molecular weight, of different chemical nature, with different binding strength, and of homogeneous or heterogeneous structure. In this patent application, these molecules are referred to by the generic name binding molecules, and since they bind a specific structure, they are called specific binding molecules. The binding molecules can be of varying structure as described above, of a peptide nature, nucleic acid nature or of another chemical nature, and they will of course have different structures for different binding specificities. In addition, they may have different types of signal-providing residues bound to them, e.g. different types of fluorescent residues. In the present patent application, different types of binding molecules may be used, both different types in the meaning different types of structures (peptides, nucleic acids or other structures) or different compositions within one type of structure (e.g. different amino acid sequences within a peptide structure, which would give different types of binding qualities) as well as different types of signal-providing residues, e.g. fluorescent residues with different types of fluorescent qualities, e.g. different excitation or emission wavelengths.

The above mentioned specific binding molecules, used for quantitation of substances in complex sample solutions, will as a rule have higher affinity for the substances to be quantified, or analogues or fragments or derivatives of said substances, than for other substances that may be present in the sample. Typically, a monoclonal antibody would have higher affinity for the substance used for immunization and selection when producing the antibodies, than for other substances in the test solution. When antibodies are used as a specific binding molecule, the substance for which the antibody has high affinity is called antigen, or hapten (the last term is often used if it is a smaller structure in a larger molecule). When the specific binding molecule is not an antibody, the term <<ligand>> is often used more than the term <<antigen>>. In this patent application, the term <<binding pair>> is used as a generic name for the molecules that are bound to each other in the specific binding reaction and the individual molecules that constitute the binding pairs will be referred to as binding partners.

Generally we can set up the following equation for the reaction between the two binding partners and the binding pairs they can form:

B1+B2=BB

where

B1 symbolizes binding partner 1 and B2 symbolizes binding partner 2, and BB can symbolize the binding pair they form.

Furthermore the equilibrium between the free binding partners and the binding pair they form, will be characterized by an equilibrium constant k (often called affinity equilibrium constant) in accordance with the law of mass action.

$$k = \frac{[ BB ]}{[ B1 ] * [ B2 ]}$$

Expressed like this, the constant is often called the affinity equilibrium constant, while the inverse form is called the dissociation equilibrium constant.

To be able to measure these binding molecules' formation of chemical binding to the analytes, one originally used a radioactive labeling of one of the binding partners. Originally, one would add a known quantity of a radioactively labeled binding partner B1 or B2, chemically identical to or near identical to the analyte, and measure this radioactively labeled substance's ability to compete with the analyte for the binding on the binding molecules that are used (originally complete immunoglobulins).

In this so-called competitive assay method the analyte molecules thus competed with labeled analogue molecules for binding molecules which were in concentration deficit. Later on, non-competitive methods, with a surplus of antibodies, were developed, where most often two different antibodies are used, one antibody to isolate the analyte (e.g. bind the analyte molecules to a solid phase), and one radioactively labeled antibody to generate a signal for measuring. This method was called the immunometric method.

Common to the competitive and the immunometric method, was the use of standardized test solutions with a known quantity of analyte to calibrate and correlate the measured signal with the concentration of analyte, and the generation of a so-called standard curve or calibration curve. Tests with unknown concentrations of analyte were determined by interpolation on this curve.

The radioactive labeling methods are still the ones most frequently used, but early on attempts were made to find non-radioactive methods for determining the presence of analyte analogues or binding molecules. The use of enzymes is signal generating molecules constituted a big step forward, as radioactivity was avoided, longer durability of the reagents was achieved, and simpler measuring equipment, typically light absorption spectrophotometers, could be used. Another big step forward was achieved when fluorescent molecules were taken into use as signal generating molecules. This improved the sensitivity compared to the pure absorption photometric methods.

Chemoluminescence methods have increased the sensitivity further.

With the use of most types of signal-generating residues, both radioactive, fluorescent and enzymatic substances, the need arose to separate the signal-generating residues bound to molecules that had been bounded to the specific binding molecules, from those residues that were not bound to said binding molecules. Typically, this could be plastic surfaces, glass surfaces, porous filters or particle based matrices with immobilized binding molecules or more unspecific media, such as e.g. active coal, which can separate unbound small-molecular a molecules from analyte-molecules bound to larger binding molecules.

It was observed early on, that antibodies in themselves could precipitate analytes, and this could be used for quantification of the analytes without any of the signal-generating attached residues being involved. Concentration determination by the help of antibody precipitation has low sensitivity, but is very practical because it is not necessary to use a solid separation phase involving a washing step, and this simplifies the execution. Precipitation analysis in gels and directly in a fluid state has lead to a more widespread use of immunoassays and made way for a high degree of automation in spectrophotometric automations such as Hitatchi-instruments and automated nephelometres which are for instance delivered by the Dade Behring company. In addition to the limited sensitivity, it has been difficult to use monoclonal antibodies in the precipitation analytic methods. It is presumed that monoclonal antibodies often only bind onto one area of the analyte molecule, whereas polyclonal antibodies usually will bind onto several places on the analyte molecules, and thus more easily lead to larger aggregations of antibodies and analyte molecules that easily precipitate. Thus in contrast to the present invention the above mentioned method does not comprise immunocomplexes, does not use fluorescence for quantification, is not a fluorescence polarisation assay and has low sensitivity.

The so-called BiaCore instruments and other technology based on plasmon resonance have further shown that direct measurement of an analyte without signal-providing residues is possible. These methods do not, however, have the same simple technical execution as the less sensitive turbidimetric methods. Plasmon resonance instruments are in addition most often very expensive instruments.

The need to measure the analytes binding to the binding molecules without using separation devices and solid phases, and also without using substrate or washing solutions, led to the so-called proximity assays:

The company SYVA in 1974 launched an immunoassay technology for small molecules, based on competition between the analyte molecules and enzyme labeled analyte analogues for binding to antibodies, where binding onto the antibodies gave a direct effect on the enzyme activity, which could be measured without separation or washing solutions. Fluorescence polarization immunoassays (FPIAs) were introduced as early as the end of the 1970's. This method has also been most successful with small-molecular analytes, but the use of competing small-molecular fluorescence labeled analyte analogues opened up for the use of the method for quantification of large-molecular analytes, such as proteins. Some proteins also have relatively mobile subunits, making possible direct fluorescence polarization immunoassay measurements) as in the U.S. Pat. No. 4,902,630 <<Fluorescence polarization immunoassay and reagent for measurement of C-reactive protein>>, by Bennet and Chiapetta, (1990). Terpetschnig, E. et al. in <<Fluorescence polarisation immunoassay of a high-molecular-weight antigen based on a long-lifetime Ru-ligand complex>>, Anal. Biochem. 227, 140-47, 1995, have further described how asymmetric ruthenium-complexes can be used for fluorescence polarization measurements of analytes with higher molecular weight. These can, however, not be used under the presence of hemoglobin or high bilirubin concentrations.

Most fluorescence polarization immunoassays are based on competitive methods, where fluorescence labeled analogues of the analyte have been added and have competed with the analyte molecules in the test solution for the specific binding molecule, typically in the form of an antibody. It has often been necessary to use a relatively high aggregate concentration of antibodies in such assays, even concentrations where one would expect competition not to occur. So most commercial assays of this kind have a quite high aggregate concentration of antibodies, and apparently competition takes place for a considerably lower efficient binding molecule concentration. No systematic literature on this is according to our to knowledge available, but a closer analysis of the prior art might show that also other commercial competitive fluorescence polarization immunoassays are based on a relatively high aggregate antibody concentration.

Already in the mid 1980's, the company Amersham introduced scintillation proximity assays (U.S. Pat. No. 4,569,649, in which a radioactive beta ray emitted from a binding partner triggers a fluorescence radiation from a fluorescent particle that is bound to the other binding partner in the binding pair in question. This technology has found a wide range of use in the search for remedies and other specific binders in a laboratory environment, but has never found much use in practical clinical diagnostics.

Already in 1976 Ullman and Schwarzberg, in U.S. Pat. No. 3,996,345, published a method for <<Fluorescence quenching with immunological pairs in immunoassays>>. This made possible fluorescence proximity assays without the use of separation and washing steps, but advanced fluorimeters were still needed, the quenching technology was never found to be competitive in clinical routine use. In 1998 Buechler & al. published U.S. Pat. No. 5,763,189 <<Fluorescence energy transfer and intramolecular energy transfer in particles using novel compounds>>, mainly based on advanced particles comprising both binding molecules and different molecules that interact with light at different wavelengths. There are considerable development cost and production costs related to such particles. This is not a homogeneous immunoassay, and the handling of the solid phase is demanding, both in production, transportation and in execution of such assays.

Common to all the above described technologies is that those reagents used are added from several reagent containers, usually using pipetting equipment, and it requires trained specialized personnel to carry this out. The need for several reagent containers and specialized pipetting equipment and specialized personnel is a considerable cost in health service.

Furthermore, several inventions are related to methods and assays detecting or quantitating organic compounds in test samples.

WO 00/16099 (Wolf) describes reduced vacancy carbohydrate binding ligands (CBLs) that can be used to detect or quantitate carbohydrates in a sample. CBL can be used with fluorescence resonance energy trander (FRET) to evaluate free carbohydrate or those within a carbohydate containing compound by using e.g. a proximity-based signal generating label moiety. Contrary to the present invention this method is not a fluorescence polarisation assay.

EP 0 561 653 A1 (Lakowicz et al.) describes determination of glucose in a sample by contacting the test sample with a donor-acceptor pair, wherein the acceptor in the donor-acceptor pair can be competitively replaced by the analyte. The donor can be photo luminizing or fluorescent. This method is suitable for higher concentrations of analyte and the method is not a fluorescence polarisation assay.

WO 00/25134 (Blanchard et al.) describes an assay for identifying ligands for nuclear receptors, utilizing scintillation proximity and FRET. Contrary to the present invention this assay is not a fluorescence polarization assay. It is using a heterodimeric partner, and is not suitable for fluorescence polarisation assay.

U.S. Pat. No. 5,814,449 (Schultz and Ballerstadt) describes a method for detection of galactose and glucose using a receptor carrying molecule with at least two binding sites for the analyte of interest. In addition there are two groups of molecules wherein one group (fluorochrome) can produce a detectable response in the proximity of the other molecule. The group of molecules is bound to an analogue of the analyte of interest. When the analyte is present the binding complex will dissociate due to competitive replacement of the analogue with the analyte. The detection is performed with a complicated sensor and contrary to the present invention this method is not a fluorescence polarisation assay.

EP 0984281 A2 (Ullman et al.) describes a photosensitizer associated with a molecule in in a specific binding pair (sbp), and a chemo luminous component associated with a sbp molecule, and wherein the amount of light emitted from the chemo luminous compound due to the activation of the photosensitive is related to the amount of analyte in the sample. This method requires several steps of pipetting and adding of reagents and is furthermore not a fluorescence polarisation assay.

Limitations in today's technology and related needs for improvements:

A considerable part of the routine analyses of tests of biological fluids, such as blood, serum, plasma, urine and spinal fluids, are carried out in so-called <<emergency>> situations, in which sending away test material to specialized laboratories causes delays before the analysis results are returned to the attending physician. Furthermore, a considerable cost problem is present in the health care system, and there is a need for efficient and at the same time sensitive reagents that are cheap to produce and simple to use. Homogeneous reagents in an ideal solution, where there are no production, storage and stability problems (contrary to with particle suspesions) are therefore a clear advantage. Furthermore, in some geographical areas there is a lack of specialized personnel and specialized laboratories, which makes it difficult to perform quality pipette and handling work, as well as necessary washing procedures and maintenance of adequate handling equipment.

There is therefore an object to provide sensitive specific measurement methods based on stabile, durable reagents, preferably in ideal solution (not in suspensions) supplied in very few and preferably just one single reagent container, not requiring any significant pipette work with no solid phase to be washed or two different phases to be separated, and which preferably can be carried out on blood tests with the presence of hemoglobin and blood cells, possibly after or with simultaneous lysis of the blood wells.

The above mentioned object is obtained by the present invention characterised by the enclosed claims.

### BRIEF DESCRIPTION OF THE INVENTION

The present invention provides a method for concentration determination of one or more analytes in a test, which is characterized by the fact that the reagent characterised by this invention is mixed with the said test solution, after which the signals generated by the fluorescent substances included in the said reagent are measured, in order to calculate the concentration of the said analytes on this basis. The said signal changes may be measured both as so-called endpoint measurements (after the establishment or near establishment of new chemical equilibriums), and kinetically (by measuring the signals' change per time unit or within a time interval).

The present invention further relates to a method where the ingredients of the said reagent is not kept separately but supplied in a single container or compartment, and the said reagent furthermore comprises at least one type of specific binding molecule for each analyte, for which the concentration should be determined, and the reagent furthermore comprises fluorescent substances whose signals change as a result of admixing a test sample with the reagent, and that this signal change is a function of the concentrations of the analyte or analytes in the sample, and that this signal change may be used to calculate the concentration or concentrations of analytes, without using separation of different states of aggregation.

The present invention relates further to a method wherein the reagent for concentration determination of one or more analytes in a test may further be characterized by the presence in the said reagent of a binding pair where the binding partners are reversibly bound to each other for each analyte to be concentration determined, and further characterized by at least one of the binding partners in each of the binding pairs having a fluorescent residue. The invention is further characterized by at least one of the binding partners in each of the said binding pairs having affinity for one of the analytes to be quantified, and that—when the reagent is mixed with the said test—each of the analytes compete for the binding between the binding partners in at least on of the binding pairs, and that varying concentration of the analyte or the analytes in the test leads to changes in the concentrations of the other molecules that are included in the equilibrium for creation of binding pairs between the said binding partners.

The invention is further characterized by the fact that the reagent may include several types of binding partners or binding pairs for each analyte.

The invention is further characterized by the fact that the reagent may be designed to quantify only one analyte and further be characterized by comprising only one type of specific binding molecule.

The invention is further characterized by the fact that the fluorescent residues may be bound to one or more of the specific binding molecules, and that the fluorescence signal that may be generated is changed as a consequence of the said specific binding molecules being bound to analyte molecules.

The invention is further characterized by the fact that the said fluorescent residues may be different fluorescent substances in order to achieve different fluorescence wavelengths to quantify different analytes in the test.

The invention is further characterized by the fact that for analyses with the presence of hemoglobin fluorescent residues with a maximum absorption coefficient at a wavelength between 600 nm and 1000 nm are preferred, more preferred exceeding 620 nm, most preferred exceeding 640 nm.

Especially preferred are reagents which ingredients are not kept separately but are present in one single container and comprise fluorescent residues bound to specific binding molecules with a low molecular weight and with fluorescent residues with a maximum absorption coefficient at a wavelength between 600 nm and 1000 nm, more preferred exceeding 620 nm, and even more preferred exceeding 640 nm.

The invention is further characterized by the reagent including specific binding molecules consisting of monoclonal or polyclonal antibodies or immunoreactive fragments of these, e.g. FAB fragments or single chain fragments or single chain antibodies, or peptides or other polymers produced by Phage Display or other biological combinatory techniques, or nucleic acid polymers or analogues or derivatives of these, or polymers produced on the basis of library technologies or synthetic combinatory chemistry. The invention may furthermore be characterized by the fact that other binding partners in said binding pair might be a derivative or analogue or fragment or part of or an imitation of the structure characterizing at least one of the analytes to be quantified.

The invention is further characterized by the fact that the reagent may include one or more types of specific binding molecules and one or more binding partners to the said specific binding molecules, and that this or these said binding partners are constituted by a fluorescent derivative of an analyte or a fluorescent analogue of an analyte or a fluorescent fragment of an analyte or a fluorescent part of an analyte or a fluorescent imitation of the structure characterized by at least one of the analytes that are to be concentration determined using the reagent.

The invention is further characterized by the fact that the said reagent may comprise lysing substances or coagulation restrainers or surface-active substances or precipitating substances or separating substances.

The invention is further characterized by the fact that the said fluorescent residues may be cyanine dyes. The invention is further characterized by the fact that Alexa Fluor Dyes or substances in the group Bodipy delivered by Molecular Probes may be used.

The invention is further characterized by the fact that it may be possible or desirable to keep parts of the reagent in separate containers, and that the reagent is ready-mixed by the user before using the reagent.

The said method is further characterized by the fact that the signals to be measured are measured using fluorescence polarization measurements. The fluorescence signals are read as a function of time, either in the form of continuous reading within a period of time or as change per time unit between 2 or more points of time or as an absolute change between 2 or more points of time. Such measurements are often called kinetic measurements or readings. The present invention may also use kinetic reading methods with the remaining forms of fluorescence measurement methods that are described.

The said method is further characterized by the fact that the different constituents of the said reagent may—if desirable—be added in steps instead of at the same time. The said method is further characterized by the fact that—if desired—more, or other, reagents may be added after the reagent characterized by this invention is added. If the reagent characterized by this invention is split up and added in steps, other reagents may—if desirable—be added in between the said stepwise adding of the reagent characterized by this invention.

The said method is further characterized by the fact that the test sample may be a biological material or extract or a dilution or concentrate, or a filtrate thereof. The said method is further characterized by the fact that the biological solutions may be blood, serum, plasma, cells from blood, lysate of blood, urine, cerebrospinal fluid, lachrymal fluid, saliva, aspirate from the gastrointestinal tract, semen or seminal fluid or feces or fecal extract or fecal dilution or suspension. The biological solution may furthermore come from the plant kingdom in the form of solutions, extracts or derivatives or filtrates.

The method for concentration determination in compliance with the invention is further characterized by the fact that standard solutions or calibrators with known concentrations of the analyte or analytes may be used, and that the concentration of the analyte or analytes is determined when the signals measured from the fluorescent residues are interpolated on the standard curve achieved using the said calibrators or standard solutions.

The method for concentration determination in compliance with the invention is further characterized by the fact that the said standard curves may be stored in an artificial memory connected to the analysis system, so that in the relevant user situation it is not necessary to perform analyses of the said calibrators or standard solutions.

The method for concentration determination in compliance with the invention is further characterized by the fact that the method may be carried out at a constant temperature, or by the use of correction algorithms empirically generated by way of studies of the temperature's influence on test solutions with a known concentration of the analyte or analytes.

According to the present invention the method may be used to determine concentrations of clinical related substances in samples of biological material from living organisms in need thereof. Such organisms may constitute plants, insects, birds, animals such as mammals, preferably primates, more preferably humans.

Furthermore the present invention is related to a kit comprising containers containing predetermined volumes of ingredients to be combined in in one single container in relation to the specific analyte to be measured, and a container for drawing specific volumes of the sample of the biological material of interest. In another embodiment the kit comprises a single container containing the reagent specific for the analyte to be measured, and a container for drawing specific volumes of the biological sample of interest.

The present invention will now be described in more detail, with reference to figures and examples.

### FIGURES

FIG. 1:

An example of concentrations as a function of time when the affinity equilibrium constant between the analyte molecules on the one side and the specific binding molecule on the other side is 10*E10/Molar, and the dissociation rate constant is 0.1 per sec.

A=The concentration of analyte molecules

B=The concentration of fluorescent binding pairs (complexes between the specific binding molecules and fluorescent derivates of analyte molecules).

C=The concentration of fluorescent derivates of analyte molecules.

D=The concentration of a specific binding molecule.

E=The concentration of binding pairs without fluorescence (complexes between specific binding molecules and analyte molecules).

Reaction diagram:

k_{affinity equilibrium}=1.0*10E10

Analyte molecule+binding molecule=non-fluorescent binding pair

k_{dissociation}=1.0*10E−1

Fluorescent binding pair=Specific binding molecule+fluorescent analyte molecule derivate

k_{affinity equilibrium}=1.0*10E10

Fluorescent analyte molecule+binding molecule=fluorescent binding pair

k_{dissociation rate}=1.0*10E−1

Non-fluorescent binding pair=specific binding molecule+analyte molecule

FIG. 2:

An example of concentrations as a function of time when the affinity equilibrium constant between the analyte molecules on the one side and the specific binding molecule on the other side is 10*E10/Molar, and the dissociation rate constant is 0.01 per sec.

A=The concentration of analyte molecules

B=The concentration of fluorescent binding pairs (complexes between the specific binding molecules and fluorescent derivates of analyte molecules).

C=The concentration of fluorescence derivates of analyte molecules.

D=The concentration of a specific binding molecule.

E=The concentration of binding pairs without fluorescence (complexes between specific binding molecules and analyte molecules).

Reaction diagram:

k_{affinity equilibrium}=1.0*10E10

Analyte molecule+binding molecule=non-fluorescent binding pair

k_{dissociation rate}=1.0*10E−2

Fluorescent binding pair=specific binding molecule+fluorescent analyte molecule derivate

k_{affinity equilibrium}=1.0*10E10

Fluorescent analyte molecule+binding molecule=fluorescent binding pair

k_{dissociation rate}=1.0*10E−2

Non-fluorescent binding pair=specific binding molecule+analyte molecule

FIG. 3:

An example of concentrations as a function of time when the affinity equilibrium constant between the analyte molecules on the one side and the specific binding molecule on the other side is 10*E10/Molar, and the dissociation rate constant is 0.001 per sec.

A=The concentration of analyte molecules

B=The concentration of fluorescent binding pairs (complexes between the specific binding molecules and fluorescent derivates of analyte molecules).

C=The concentration of fluorescent derivates of analyte molecules.

D=The concentration of a specific binding molecule.

E=The concentration of binding pairs without fluorescence (complexes between specific binding molecules and analyte molecules).

Reaction diagram:

k_{affinity equilibrium}=1.0*10E10

Analyte molecule+binding molecule=non-fluorescent binding pair

k_{dissociation rate}=1.0*10E−3

Fluorescent binding pair=specific binding molecule+fluorescent analyte molecule derivate

k_{affinity equilibrium}=1.0*10E10

Fluorescent analyte molecule+binding molecule=fluorescent binding pair

k_{dissociation rate=}1.0*10E−3

Non-fluorescent binding pair=specific binding molecule+analyte molecule

FIG. 4:

An example of concentrations as a function of time when the affinity equilibrium constant between the analyte molecules on the one side and the specific binding molecule on the other side is 10*E11/Molar, and the dissociation rate constant is 0.01 per sec.

A=The concentration of analyte molecules

B=The concentration of fluorescent binding pairs (complexes between the specific binding molecules and fluorescent derivates of analyte molecules).

C=The concentration of fluorescent derivates of analyte molecules.

D=The concentration of a specific binding molecule.

E=The concentration of binding pairs without fluorescence (complexes between specific binding molecules and analyte molecules).

Reaction diagram:

k_{affinity equilibrium}=1.0*10E11

Analyte molecule+binding molecule=non-fluorescent binding pair

k_{dissociation rate}=1.0*10E−1

Fluorescent binding pair=specific binding molecule+fluorescent analyte molecule derivate

k_{affinity equilibrium}=1.0*10E11

Fluorescent analyte molecule+binding molecule=fluorescent binding pair

k_{dissociation rate}=1.0*10E−1

Non-fluorescent binding pair=specific binding molecule+analyte molecule

FIG. 5:

An example of concentrations as a function of time when the affinity equilibrium constant between the analyte molecules on the one side and the specific binding molecule on the other side is 10*E11/Molar, and the dissociation rate constant is 0.01 per sec.

A=The concentration of analyte molecules

B=The concentration of fluorescent binding pairs (complexes between the specific binding molecules and fluorescent derivates of a molecules).

C=The concentration of fluorescent derivates of analyte molecules.

D=The concentration of a specific binding molecule.

E=The concentration of binding pairs without fluorescence (complexes between specific binding molecules and analyte molecules).

Reaction diagram:

k_{affinity equilibrium}=1.0*10E11

Analyte molecule+binding molecule=non-fluorescent binding pair

k_{dissociation rate}=1.0*10E−2

Fluorescent binding pair=specific binding molecule+fluorescent analyte molecule derivate

k_{affinity equilibrium}=1.0*10E11

Fluorescent analyte molecule+binding molecule=fluorescent binding pair

k_{dissociation rate}=1.0*10E−2

Non-fluorescent binding pair=specific binding molecule+analyte molecule

FIG. 6:

A sample of concentrations as a function of time when the affinity equilibrium constant between the analyte molecules on the one side and the specific binding molecule on the other side is 10*E11/Molar, and the dissociation rate constant is 0.001 per sec.

A=The concentration of analyte molecules

B=The concentration of fluorescent binding pairs (complexes between the specific binding molecules and fluorescent derivates of analyte molecules).

C=The concentration of fluorescent derivates of analyte molecules.

D=The concentration of a specific binding molecule.

E=The concentration of binding pairs without fluorescence (complexes between specific binding molecules and analyte molecules).

Reaction diagram:

k_{affinity equilibrium}=1.0*10E11

Analyte molecule+binding molecule=non-fluorescent binding pair

k_{dissociation rate}=1.0*10E−3

Fluorescent binding pair=specific binding molecule+fluorescent analyte molecule derivate

k_{affinity equilibrium}=1.0*10E11

Fluorescent analyte molecule+binding molecule−fluorescent binding pair

k_{dissociation rate}=1.0*10E−3

Non-fluorescent binding pair=specific binding molecule+analyte molecule

FIG. 7:

An example of concentrations as a function of time when the affinity equilibrium constant between the analyte molecules on the one side and the specific binding molecule on the other side is 5*10E9/Molar, and the dissociation rate constant is 0.1 per sec.

A=The concentration of analyte molecules

B=The concentration of fluorescent binding pairs (complexes between the specific binding molecules and fluorescent derivates of analyte molecules).

C=The concentration of fluorescent derivates of analyte molecules.

D=The concentration of a specific binding molecule.

E=The concentration of binding pairs without fluorescence (complexes between specific binding molecules and analyte molecules).

Reaction diagram:

k_{affinity equilibrium}=5.0*10E9

Analyte molecule+binding molecule=non-fluorescent binding pair

k_{dissociation rate}=1.0*10E−1

Fluorescent binding pair=specific binding molecule+fluorescent analyte molecule derivate

k_{affinity equilibrium}=5.0*10E9

Fluorescent analyte molecule+binding molecule=fluorescent binding pair

k_{dissociation rate}=1.0*10E−1

Non-fluorescent binding pair=specific binding molecule+analyte molecule

FIG. 8:

An example of concentrations as a function of time when the affinity equilibrium constant between the analyte molecules on the one side and the specific binding molecule on the other side is 5*10E9/Molar, and the dissociation rate constant is 0.01 per sec.

A=The concentration of analyte molecules

B=The concentration of fluorescent binding pairs (complexes between the specific binding molecules and fluorescent derivates of analyte molecules).

C=The concentration of fluorescent derivates of analyte molecules.

D=The concentration of a specific binding molecule.

E=The concentration of binding pairs without fluorescence (complexes between specific binding molecules and analyte molecules).

Reaction diagram:

k_{affinity equilibrium}=5.0*10E9

Analyte molecule+binding molecule=non-fluorescent binding pair

k_{dissociation rate}=1.0*10E−2

Fluorescent binding pair=specific binding molecule+fluorescent analyte molecule derivate

k_{affinity equilibrium}=5.0*10E9

Fluorescent analyte molecule+binding molecule=fluorescent binding pair

k_{dissociation rate}=1.0*10E−2

Non-fluorescent binding pair=specific binding molecule+analyte molecule

FIG. 9:

An example of concentrations as a function of time when the affinity equilibrium constant between the analyte molecules on the one side and the specific binding molecule on the other side is 5*10E9/Molar; and the dissociation rate constant is 0.001 per sec.

A=The concentration of analyte molecules

B=The concentration of fluorescent binding pairs (complexes between the specific binding molecules and fluorescent derivates of analyte molecules).

C=The concentration of fluorescent derivates of analyte molecules.

D=The concentration of a specific binding molecule.

E=The concentration of binding pairs without fluorescence (complexes between specific binding molecules and analyte molecules).

Reaction diagram:

k_{affinity equilibrium}=5.0*10E9

Analyte molecule+binding molecule=non-fluorescent binding pair

k_{dissociation rate}=1.0*10E−3

Fluorescent binding pair=specific binding molecule+fluorescent analyte molecule derivate

k_{affinity equilibrium}=5.0*10E9

Fluorescent analyte molecule+binding molecule=fluorescent binding pair

k_{dissociation rate}=1.0*10E−3

Non-fluorescent binding pair=specific binding molecule+analyte molecule.

Chemical analyses of materials are most often performed by taking a sample of the material (i.e. test material/test sample), which subsequently undergoes the desired chemical analysis. The test sample may be e.g. in the fluid state, gaseous state, solid state or mixtures of the said states of aggregation. The sample can furthermore be homogeneous or inhomogeneous. If, for instance, the sample is in a fluid phase, the fluid phase could comprise particulate material, and would thus be inhomogeneous. Well-prepared blood serum is an example of a homogeneous biological sample material, whereas the blood as such comprises considerable amounts of blood cells, and thus is not a homogeneous material.

Concentration determination of one or more analytes in a test sample is most often achieved by admixing other chemical substances to the sample, after which the signals or signal changes appearing as a consequence of the mixing are observed or measured. These signals may be chemical or physical signals in the form of electromagnetic radiation, radioactive radiation, temperature or response to physical influence, such as fluorescence or absorption of light. In previously known commercial products for quantitative analysis using fluorescence measurements, typically in the form of immunoassay systems, the binding partners included in binding pairs, typically in the form of antibodies and analyte analogues labeled with fluorescent residues, have been kept isolated from each other, typically separated in different reagent containers. This have made it necessary to add different reagents in several steps by the use of accurate instruments, e.g. pipettes.

In order to avoid the different problems that are connected to the currently used technology, the present invention provides all the necessary chemical substances, including fluorescent substances and binding partners, in one single collection of substances, usually in a fluid state, and gathered in one single container. This collection of chemical substances is called a reagent. The reason why it is an advantage to have one single ready mixture of the chemical substances in one reagent is that this provides a pre-measured amount of correct concentrations of the ingredients. This means that persons without specialized chemical training can perform the mixing of the reagent with the test material. Typically, the test material will be collected in a pre-calibrated capillary or another testing device, e.g. a self-calibrating constant volume pipette e.g. from Samco Scientific (USA), which is subsequently emptied into or combined with (e.g. dropped into) the reagent in its container. The container with the reagent may consist of different materials, such as e.g. glass vials, glass or plastic test tubes, plastic containers, foil pockets, plastic pads or other devices that can be used to contain reagents.

Traditionally, the wish has been that the binding between the binding partners in analysis systems using binding pairs and fluorescence measurements should be as strong as possible. High affinity equilibrium constants have led to very high sensitivities for the concentration determinations, and ensured less interference from other substances and variations in the physical/chemical circumstances. In the earlier state of the art, keeping binding partners in the same reagent and reagent container has—due to the high affinity equilibrium constants—led to aggregations, precipitations and irreversible chemical changes, as well as inhomogeneous solutions. Thus, keeping them in one single reagent, as is the case according to the present invention, has therefore been avoided, and several separate reagents has been preferred. The mixing of two or more binding partners has instead been carried out during the performance of the quantitation method. To achieve a precise result, precise transfer of volumes of the reagents has therefore been necessary, either by manual so-called pipetting or using chemical automations. Using manual pipettes is very demanding and is an essential part of the basic training for laboratory workers and chemical engineers. The automations are expensive and usually only available in laboratories or larger centers for testing or handling of samples. To avoid such problems, the method according to the present invention use only one reagent for concentration determination of one or more analytes in a sample, wherein the said reagent is not kept separately but is present in one single container. Furthermore, the reagent to be used in the method according to the present invention comprises at least one specific binding molecule for each analyte to be concentration determined, as well as fluorescent substances whose fluorescence changes as a result of mixing in a sample to the reagent. This change in the fluorescence signal is a function of the concentrations of the analyte or analytes in the sample, and can be used for calculation of the analyte concentration(s) without using separation of different states of aggregation.

Thus, the generic term <<reagent>> is used in this description as a generic name for the collection of substances that are mixed with a sample solution for measuring one or more analytes. The reagent will normally be in a fluidal state, in the form of a solution of several substances, readily with buffer substances, salt, surface-active agents and anti-biological substances added to avoid growth of microorganisms in the reagent. But for some uses of the reagent, it may be an advantage if the reagent is used in a solid state, possibly by adding fluid immediately prior to using the reagent, or possibly by dissolving the solid reagent, for instance in the sample material, prior to or during use. The reagent may further be dried on a stand or a device, or enclosed in capsules or tablets.

The preferred embodiment of the reagent according to the present invention, is that the reagent is present, ready for use, in one single container where the amount of reagent that is to be used to analyze a sample will not need to be pipetted or meted out or mixed with other reagents before use.

As an alternate, however, the appropriate volume may be meted out prior to the analysis or in the course of the execution of the analysis. Thus, it is normally less preferred, but still absolutely possible, to keep parts of the reagent in one or more separate containers, so that the ready-for-use reagent is mixed prior to or immediately prior to or in connection with the execution of the analysis. If so desired, parts of the reagent may be impregnated onto or into containers or devices or filters etc. and be mobilized at contact with a solution, e.g. the assay solution. If so desired, the entirety or parts of the reagent may be in a dry or desiccated state, and if desired, it can be designed so that fluid can be added to it prior to, immediately prior to or in connection with the quantitative analysis. The reagent may then further be mixed ready-for-use by such analysis automations that are often used by larger, more sophisticated laboratories. However it to important to note that the methode according to the present invention, by combining a single reagent and fluorescence polarisation assay for analysis of analytes in samples of complex materials, distinguishes itself from the previous state of the art; e.g. from turbidimetry and nephelometry by being a more sensitive method, from scintillation proximity by the use of non-radioactive substances only and from FRET by employing ideal solutions which are less expensive and less complicated to produce.

The reagent to be used according to the present invention comprises fluorescent residues bound to (same or different) specific binding molecules/binding partners with a low molecular weight. It is known to the skilled man of the art that the ability to conserve the polarization of the exciting light in the emitted light as well, is a function of the rotation speed of the molecules, which in turn is a function of the molecular size as small molecules rotate faster than larger molecules. Thus, an especially preferred embodiment of the reagent in accordance with the present invention, is therefore to bind the fluorescent signal-providing substance to one or more binding partners with a low molecular weight, preferably a molecular weight under 5000, more preferred under 3000 and even more preferred under 1500. When the fluorescence-labeled binding partner either dissociates from its binding partner or binds to its binding partner, the total molecular size for the molecule that is rotating with the fluorescent residue will change, and this can be detected as a change in fluorescence polarization.

It is however, and according to another embodiment of the present invention, possible to use a reagent wherein the fluorescent binding partner(s) (i.e. binding partner(s) with fluorescent residue(s) bound to them) has higher molecular weight than the above mentioned, and to still use fluorescence polarisation assay in order to detect an analyte/analytes (see example 15). However, in this case the fluorescent residue(s) must display longer decay time when compared to the fluorescent residue(s) bound to specific binding molecules with low molecular weight.

The fluorescence-labeled binding molecules in the reagent that are used according to the preferred embodiment of the present method (i.e. the use of binding partner(s) with low molecular weight) are, according to the competitive embodiment of the present invention, either analogues or fragments or derivatives of the analyte(s) to be determined, whereas they in the non-competitive embodiment of the present invention are binding molecules such as a peptide/peptides or synthetic binders (optionally being identified by combinatory chemistry techniques or phage display or nucleic acid selection technology) with specific affinity for one or more of the said analytes. Furthermore, the fluorescent residues that are bound to the specific binding molecules that are used in the reagent according to the non-competitive embodiment of the present invention, preferably have a maximum absorption coefficient at a wavelength exceeding between 600 nm and 1000 nm, further preferred exceeding 620 nm, and even further preferred exceeding 640 nm.

According to the present invention the test sample solution may be a biological material or extracts thereof, such as e.g. blood, blood serum or blood plasma, lachrymal fluid, extracts of feces, plant extracts, aspirate from the gastrointestinal tract or semen or seminal fluids, possibly diluted in diluent solutions or depository solutions, possibly with other reagents added to prevent coagulation or microbiological growth or oxidation or reduction or to regulate the acidity, alternatively derivatives or filtrates.

The different embodiments of present invention may be influenced by changes in the surrounding temperature or in the test sample solution or the reagent or in the mixture or in the instrument or the measurement compartment of the instrument to be used. Such temperature influences can be counteracted by the reagents and/or the instrument being temperature regulated, or by using calibrators with known concentrations of the analyte. However, one of the aims of the present invention is to provide measurement methods where calibrators are not used. Empirical measurements and theoretical calculations based on measurements with the reagents provided by the present invention may, however, form the basis for estimated deviation as a result of varying temperatures. Thus, according to the present invention and if desired, it is possible to use combinations of temperature sensors and software connected to or as a part of the measurement instrument, providing total or partial correction for the temperature-provoked deviation in the concentration determinations.

With the use of the present invention a large number of analytes can be concentration determined. The following analytes can be listed tabularly, but there is obviously a large umber of other analytes that have not been included in this listing:

Hemoglobin

Albumin

C-reactive protein

Albumin in urine

Glycated albumin

Glycated hemoglobin

Ferritin

ASAT

ALAT

LDH

Myoglobin

Troponin I

Fatty Acid Binding Protein

Amylase

Glucose

HCG

U-HCG

TA-Tests

Insulin

Anti-insulin antibodies

Helicobacter antibodies

Thyroxin

Free ryoxin

Prostate specific antigen

Free Prostate specific antigen

Thyroid stimulating hormone

Creatine kinase type MB

These and a large number of other analytes are referred to in reference books, such as e.g. in Tietz Textbook of Clinical Chemistry, Saunders Company, ISBN 0-7216-4472-4, 1994, and others, and also later text books and general surveys in clinical chemistry and pathology.

According to the competing embodiment of the present invention, a ready-to-use preformed reagent, comprising one or more binding pairs where both binding partners in each binding pair is present in one single container, and where the reagent is composed in such a way that aggregations, precipitations and irreversible binding between the binding pairs are avoided, is used. When the test material is mixed with the reagent, the analyte(s) compete with the binding between the binding partners already present in the reagent in at least one of the binding pairs.

The reagent provided for the competitive embodiment of the present invention, can be used for concentration determination of one or more analytes, and may be characterized by the fact that for each analyte the reagent comprises at least one binding pair in which the binding partners are reversibly bound to each other. Furthermore, the reagent related to this embodiment of the invention may be characterized by at least one of the binding partners in at least one of the said binding pairs comprising or having bound to it a fluorescent chemical residue.

By <<reversibly bound>> what is meant here is a binding that is such that addition of a substance that competes with the binding between the binding partners leads to a change in the concentrations of the other molecules included in the equilibrium for formation of binding pairs between said binding partners within a reusable amount of time—typically within one hour after adding. The invention is further characterized by the said changed concentrations of the molecules included in the equilibrium for formation of binding pairs between the binding partners leading to a change in the fluorescence signals that can be generated from the said fluorescent residues. This can occur because in the reagent characterized by this invention such fluorescent residues are used which can generate signals that change when the fluorescent residue is part of a connected binding pair as compared to the signals generated when the residue is bound to a binding partner molecule that is not bound to its binding partner molecules.

The reagent used according to the competitive embodiment of the present invention is characterized by comprising one or more binding pairs for which there is, furthermore, an equilibrium between the free condition in which the binding partners are not bound to each other, and the bound condition in which the binding pairs are bound to each other. This equilibrium is subject to general chemical laws, such as the law of mass action. When the concentrations of one of the molecules included in the chemical equilibrium are changed, the concentrations of the remaining molecules included in the equilibrium will change as well. Furthermore, the equilibrium will shift by adding analogues of the binding partners or derivatives or analogues of binding partners, where the structural similarity is adequate to bring about competition over the binding to the corresponding binding partner. Such structural similarity is mostly conserved, also when modifications are used for attaching fluorescent residues that do not dramatically change the structure of the substance that is attached onto this fluorescent residue. It is further commonly known to the skilled man of the art that if fluorescent residues or labels with different fluorescence are used, it is possible to simultaneously measure different chemical reactions in the same solution. Thus, the reagent in accordance with the present invention is further characterized by the fact that by using residues or labels with different fluorescence, it can be used to quantify different analytes simultaneously in the same sample.

Traditionally, as high affinity equilibrium constant as possible was desired for the use of bio specific binders such as antibodies. The affinity equilibrium constant is a complex quantity, constituted by the association velocity constant divided by the dissociation velocity constant, and traditionally affinity equilibrium constants exceeding 10E7 have been desired, preferably exceeding 10E8 and even more preferred exceeding 10E9. At especially low concentrations affinity equilibrium constants exceeding 10E10 have been desired in order to achieve that it should be possible to bind an acceptable share of the analyte to the binder. Especially, it has been claimed that high dissociation velocity rust be avoided. Polyclonal antibodies have—since they are polyclonal—very varying affinity equilibrium constant in the same preparation, whereas monoclonal antibodies, or the biological or synthetic binders accounted for in the background for this invention, have more uniform or identical affinity equilibrium constants within the same preparation, and also often a more constant affinity from preparation to preparation. In the competitive embodiment of the present invention, and contrary to what was previously desired, especially high affinity equilibrium constants are not desired, and in particular not too low dissociation velocity constants.

As accounted for in the paragraph on the background for the present invention, the average skilled man of the art is used to using different types of specific binding molecules. The reagent in accordance with the competitive embodiment of the present invention may comprise binding partners that form binding pairs of all types and varieties. Traditional polyclonal antibodies on the one hand at antigens on the other hand may be used as specific binding molecules. Instead of polyclonal antibodies monoclonal antibodies may be used. The antibodies may be complete or in the form of reactive fragments. Especially preferred are smaller fragments of antibodies such as FAB fragments or single chain antibodies or single chain antibody fragments. Instead of antibodies or antibody fragments produced in eucaryotic cells, it is possible to use binders provided through phage display or further advancements of phage display technology, in the form of polypeptides or other types of polymers, polynucleic acids, or binders composed of building blocks that were variedly composed and picked out using library technology. Synthetic combinatory chemistry is rapidly developing and can be used to produce specific binding molecules, and production of polymers with RNA or DNA or analogue monomers is used with increasing frequency, and may of course also be used as specific binding molecules in the present intention.

The antigens mentioned above are traditional binding partners in immunoassay technology, but in recent years parts of antigens, antigen fragments, so-called haptens and derivatives of antigens or haptens, have been used to a greater extent.

Synthetically or biologically produced molecules with a high structural similarity to the analyte or analytes that are to be quantized, can also be used. The present invention is characterized by the fact that all these structures may be used in binding pairs, when a suitable binding partner is found.

The present invention may further be characterized by the use of binding pairs in the preformed reagent for which the binding of the binding partners is influenced by the concentration of the analyte or analytes. A high concentration of an analyte will lead to an increased competition for binding to one of the binding partners in one or more binding pairs, compared to what would be the case with a lower concentration of said analyte. The present invention is further characterized by the fact that this leads to another concentration of one or more of the unbound binding partners and/or the binding pairs that were present in the reagent before it was mixed with the sample material. The reagent in accordance with the present invention is further characterized by the fact that it is composed in such a way that this changed concentration of one or more of the binding partners can be detected using one or more of the methods that are described above.

In order to measure an analyte in a competitive binding assay, the binding partner that the competition is about, e.g. the specific antibody, must be in effective relative deficit in relation to the concentration of the analyte (in order for competition to arise). But since what is desired is to measure the signal that is changed as analyte molecules with fluorescent residue are bound to the binding partner, the concentration of the binding partner (e.g. antibody) must be so high that a considerable part of the analyte molecules can be bound to the binding partner. Here it is necessary to find a practical balance that is regulated by the analyte concentration in the solution and the choice of specific binding molecule with suitable affinity equilibrium consent and dissociation velocity constant. Furthermore, according to the competitive embodiment of the present invention, the binding molecules and fluorescent derivatives or analogues of the analyte molecules are kept as binding pairs in the same reagent container. Thus the use of polyclonal antibodies as specific binding molecules in combination with large molecular analytes should be avoided, since this typically could result in precipitation in the reagent or the assay solution. Monoclonal antibodies are most often to be preferred over polyclonal antibodies, and often further preferred are monovalent binding molecules readily of smaller molecular size, e.g. FAB-fragments of antibodies or polypeptides or aptamers.

When a reagent in accordance with the competitive embodiments of the present invention is mixed with a sample containing the analytes that are to be concentration determined, the concentration of the analytes in the sample material will lead to changes in the concentrations of the other molecules included in the equilibriums for formation of said binding pairs between said binding partners. The invention is further characterized by the fact that the said changed concentrations of the molecules included in the equilibrium for formation of binding pairs between the binding partners lead to a change in the signals that can be generated from the said fluorescent residues, and that these signal changes can be used for quantification of said analytes.

The said changes in the fluorescence signals are, in other words, a direct function of the concentrations of the analytes, and these signal changes can be used for concentration determination of the said analytes. Thus, the fluorescent residues according to the present invention may have one or more of the specific binding molecules bound to them, and the fluorescence signal that can be generated changes as a consequence of the said specific binding molecules binding to the analyte molecules.

If a concentration of approximately 1.0*10E−9 molar analyte is presence in the assay solution (the solution arrived at when the reagent related to the present invention is mixed with the test material), and we have an infinitely high affinity equilibrium constant, we will, by using an effective concentration of 0.5*10E−9 molar specific binding molecule, achieve 50% binding of the analyte to the binding partner at equilibrium. If the affinity equilibrium constant is lower, e.g. 1.0*10E−9, the situation at equilibrium is somewhat different:

Given that the analyze molecules have the same affinity for the binding molecules (e.g. antibodies) whether they have fluorescent residues bound to them or not, and

a=unity equilibrium constant,

b=aggregate concentration of binding partner molecule (bound and free put together), and

c=aggregate concentration analyte molecules (totally from the sample and the reagent related to the present invention, including analyte molecules with fluorescent residues),

according to the law of mass action the aggregate concentration of binding pairs (e.g. antibody-antigen complexes)=x would be as follows at equilibrium:

x=a(b−x)(c−x)

In a situation where a=1.0*10E9, b=1.0*10E−10 and c=2.0*10E−9, calculation shows that at equilibrium

x=0.7*10E−10 molar, and further that the effective concentration of free specific binding molecule (e.g. antibody) is 0.3*10E−10 molar and further that the concentration of unbound analyte molecules (with and without fluorescent residues bound to them) is 19.3*10E−10 molar.

We see that this will give a very signal change in the signal as a consequence of the analyte molecules' binding to the binding partner. In this case it would be possible to bind less than 5% of the analyte molecules to the binding partner.

If the affinity equilibrium constant a is 1.0*10E8 but b and c above are unchanged, it could accordingly be shown that the fraction of bound analyte molecule is infinitesimally small, and no competition what so ever occurs.

If the situation is such that the specific binding molecule's (e.g. the antibody's) affinity for modified analyte molecules with fluorescent residue bound to them is higher than for unmodified analyte molecules, a lower concentration of the modified analyte molecules needs to be used for the analyte molecules in the sample to be able to efficiently compete. This corresponds to a situation with a lower effective concentration of specific binding molecule, and will require a somewhat higher amity equilibrium constant of the specific binding molecule according to the law of mass action.

If the situation is such that the specific binding molecule's (e.g. the antibody's) affinity equilibrium constant for modified analyte molecules with fluorescent residue bound to them is lower than for unmodified analyte molecules, it might be desirable to use a higher concentration of the modified analyte molecules. To achieve a situation with competition, however, the effective concentration of specific binding molecule must be in molar deficiency relative to the analyte molecules in the sample, and this low concentration of the specific binding molecule still requires that the affinity equilibrium constant between binding partner and analyte molecules is high. It is worth mentioning, however, that the effective concentration of binding molecules may be considerably less than the total amount of so-called binding molecules. It is not seldom than only a fraction of the actual binding molecules—e.g. monoclonal antibodies—really bind its binding partners. This can be due to structural alterations, e.g. in the synthesis of the molecules, e.g. post-translation alterations, or modifications introduced by labeling chemistries or simply of unknown origin.

Thus we can conclude that the affinity equilibrium constant, according to a competitive embodiment of the present invention, must have a value that at least equals one third of the inverse value of the concentration of the modified analyte molecules with signal providing residues bound to them. More preferred are affinity equilibrium constants that are higher than the inverse value of the concentration of the modified analyte molecules, and even more preferred are affinity equilibrium constants higher than twice the inverse value of the concentration of the modified analyte molecules. Thus, in the reagent provided according to the competitive embodiment of the present invention, an equilibrium between the binding partners bound to each other and the binding partners in free, non-bound form will be established. When the said reagent is added to a sample, the concentrations change, and a new equilibrium is established. In the earlier technical state, a high affinity equilibrium constant and an especially high association constant was desired so that the equilibrium could be established soon after adding a binding molecule, e.g. a antibody. In the competitive embodiment of the present invention binding pairs have formed in the reagent before the sample is added, and then a higher dissociation velocity constant is sooner desired, so that a new equilibrium can be achieved within reasonable time after the apple has been added.

If affinity equilibrium constant

a=1.0*10E10/mol,

b=aggregate concentration of binder (e.g. antibody)=1.0*10E−9 molar

c=aggregate concentration analyte molecules (with and without signal providing residues)=1.0*10E−9 molar

it can, by using mass balance and the law of mass action, be shown that at equilibrium the concentration of complex between the binding partners (analyte molecules with or without signal-providing residue and binder such as e.g. antibody)=0.73*10E−9 molar, the concentration of analyte molecules unbound to binding partner=0.27*10E−9 molar, and the concentration of unbound specific binding molecules (e.g. antibody)=0.27*10E−9 molar. If then a test sample that adds 1.0*10E−9 molar analyte molecules is added, so that the total amount of analyte molecules in the assay solution becomes 2.0*10E−9 molar, a new equilibrium will be etablished after a while.

In these examples it is assumed that adding a sample does not change the total volume of the assay solution considerably. Calculations can be made showing that even though the assay solution is slightly changed, the above calculation examples will be very close to the conditions described.

If the affinity equilibrium constant is a power of 10 higher, the situation is as follows:

If affinity equilibrium constant

a=1.0*10E11/Molar.

b=aggregate concentration binder (e.g. antibody)=1.0*10E−9 Molar

c=aggregate concentration analyte molecule (with and without signal providing residues)−1.0*10E−9 molar

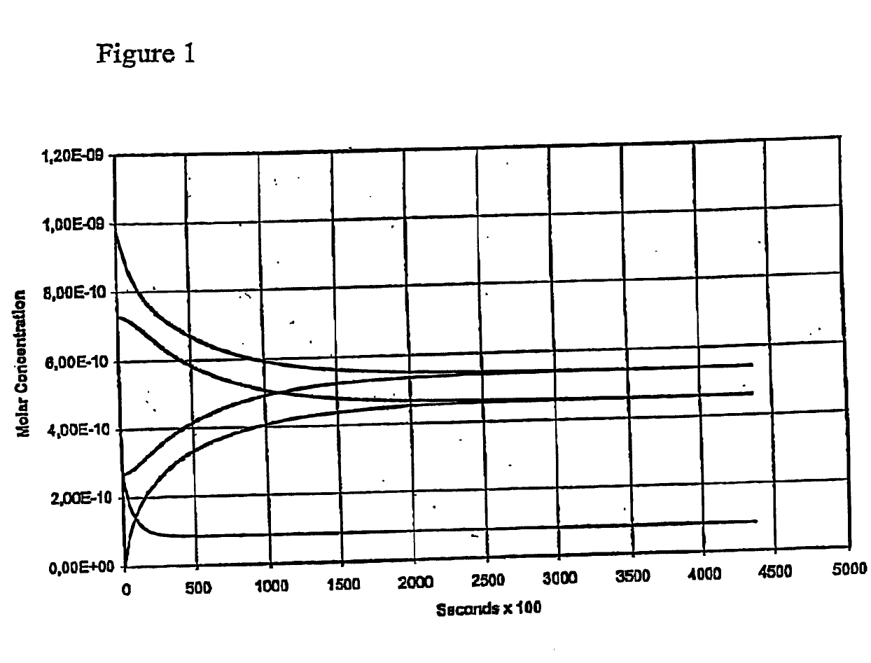

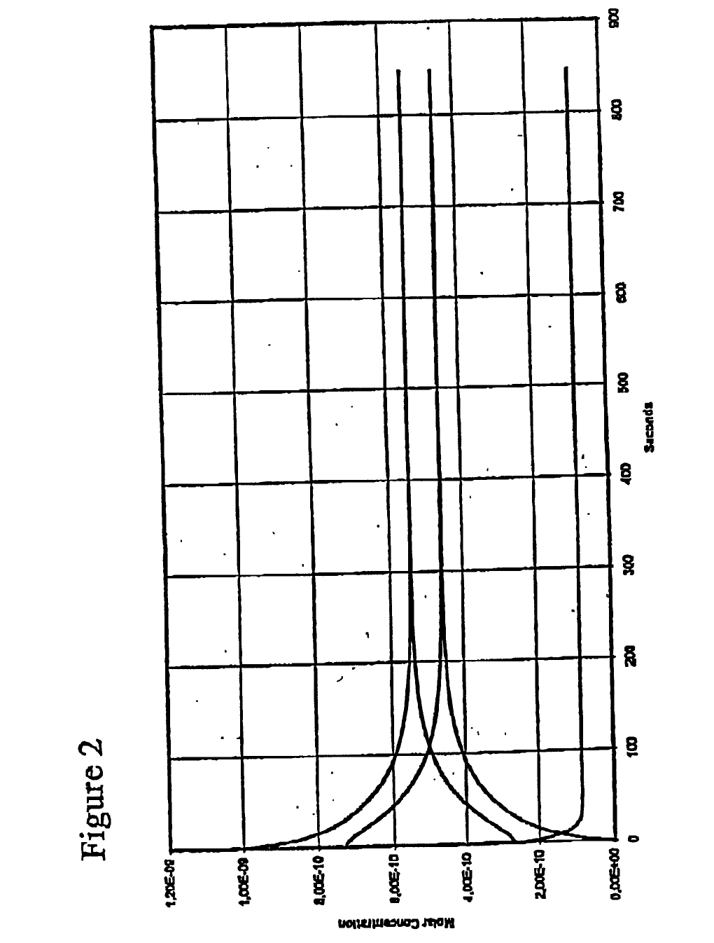

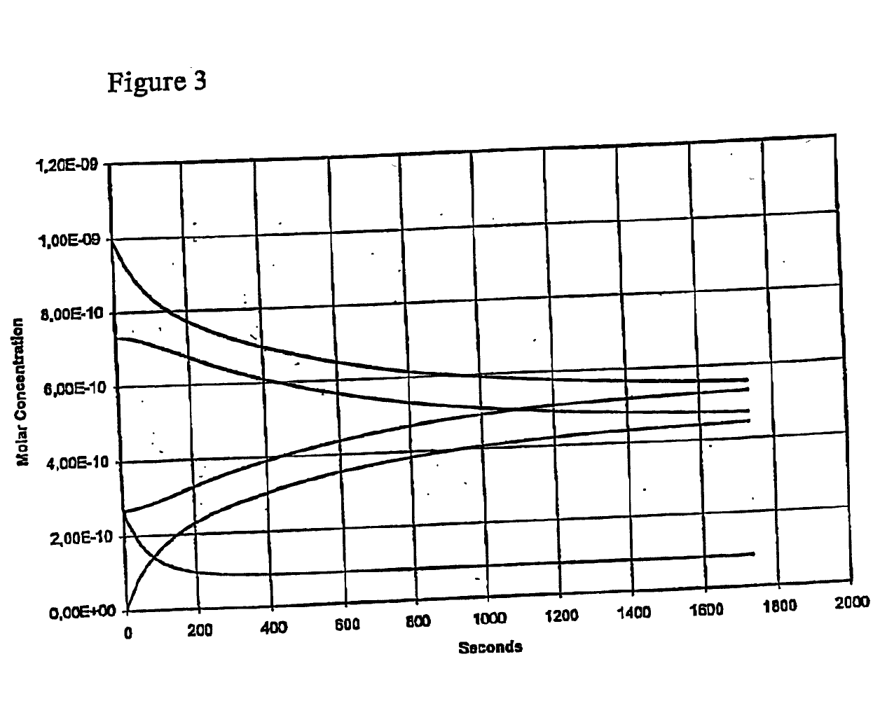

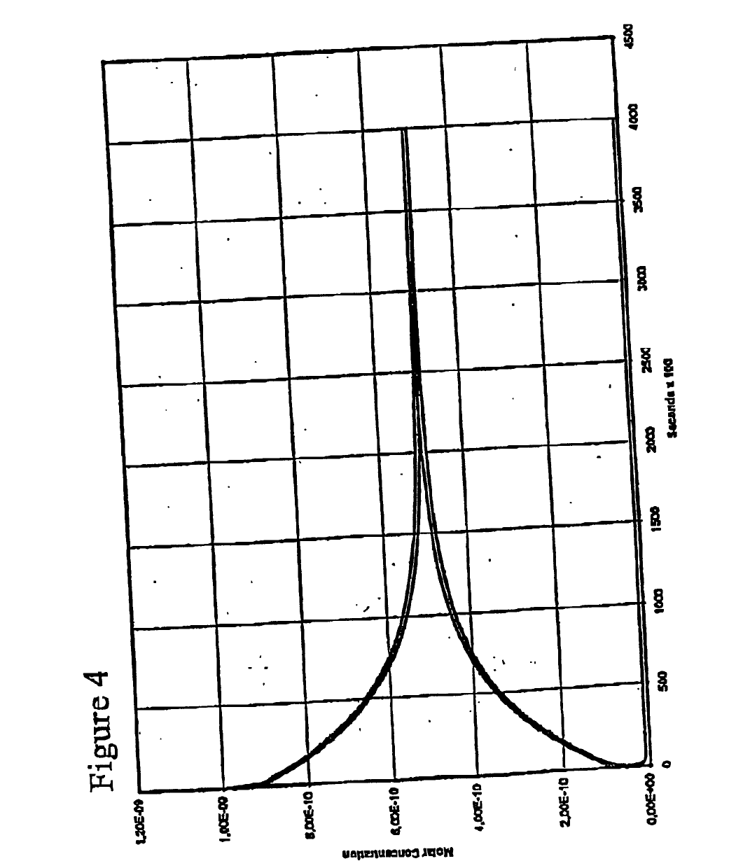

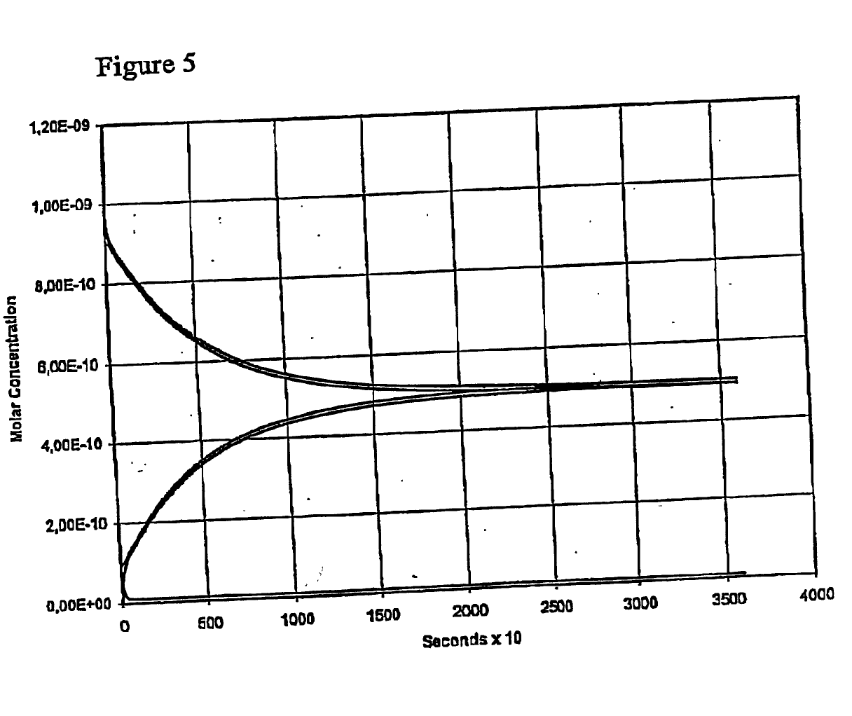

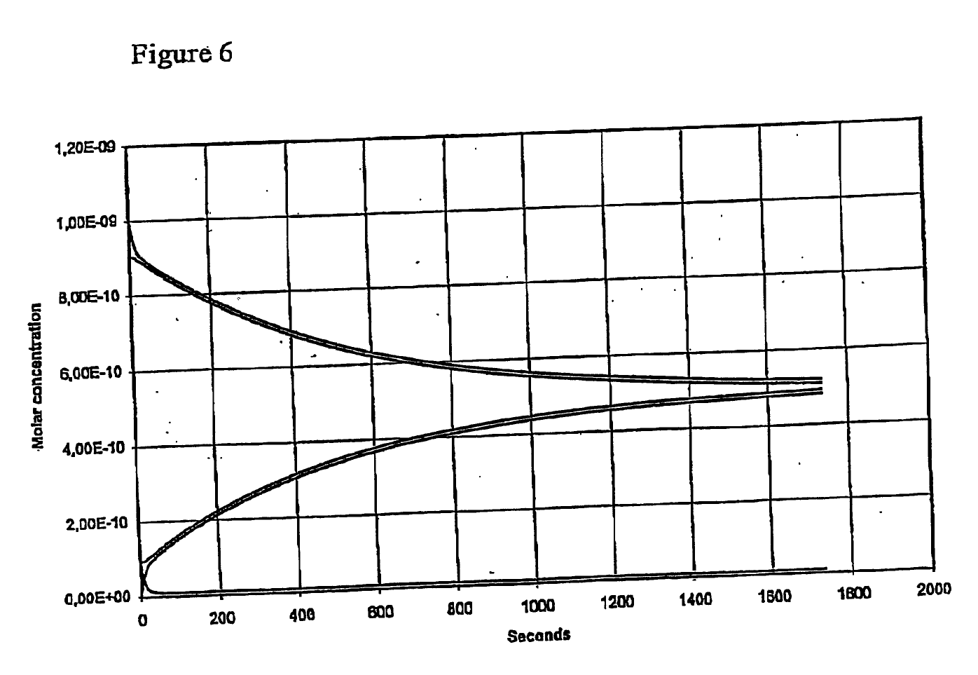

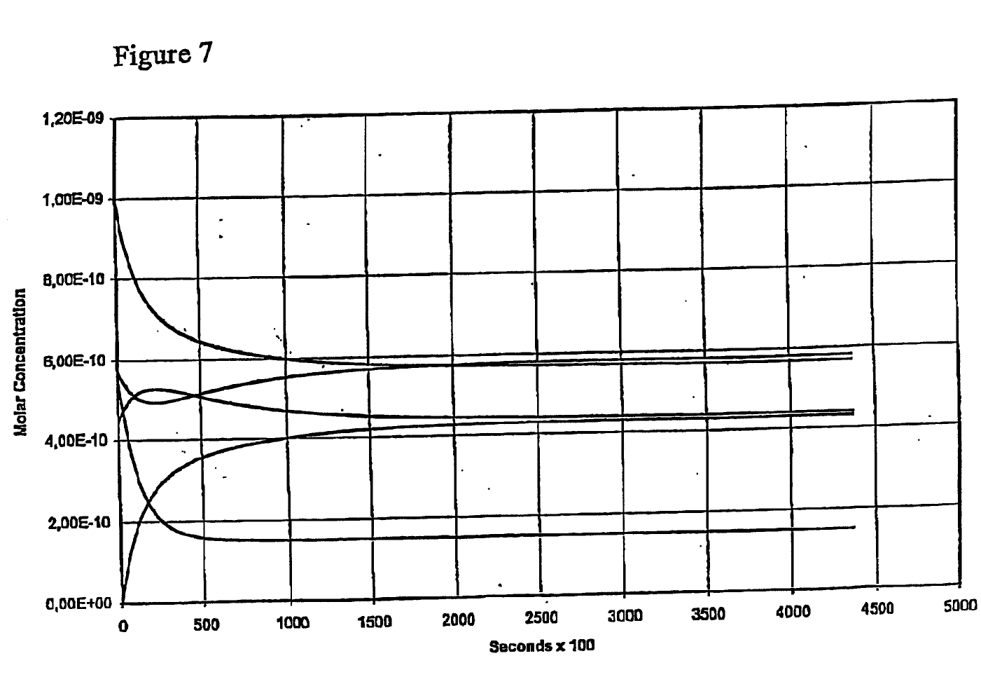

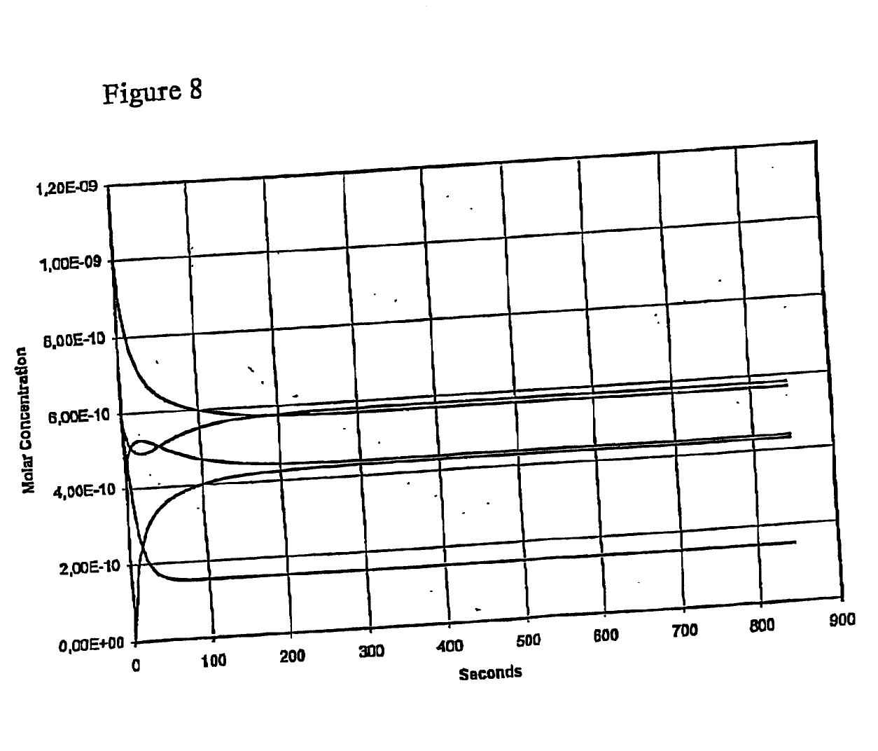

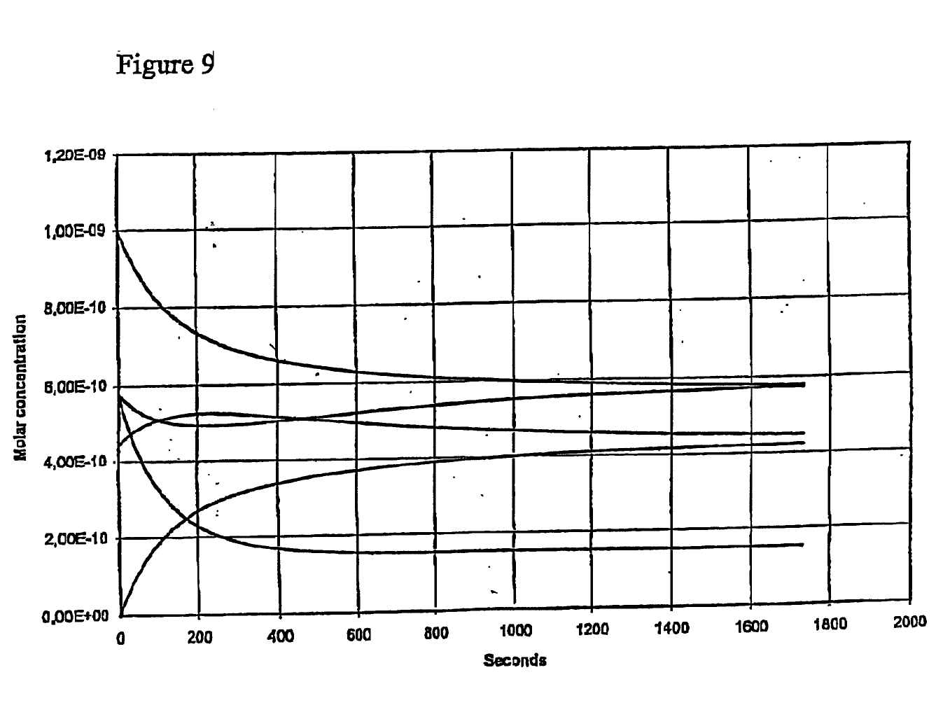

it can, using calculations corresponding to the above, be shown that in the competitive embodiment of the invention, it is predominantly the dissociation velocity constants that determines the time it takes to achieve equilibrium or near equilibrium after adding a sample. Slightly simplified we can say that FIGS. 1 to 9 show that the value of the affinity equilibrium constant is the most important factor in deciding the range of signal change that can be achieved, whilst it is the value of the dissociation velocity constant that determines how quickly a new equilibrium or near equilibrium can be achieved after mixing in a sample comprising analyte molecules, and thus how quickly a new stabile fluorescence signal is achieved. In the reagent used for endpoint measurement in the competitive embodiment of the present invention, binding pairs with dissociation velocity constants 0.003 per second are therefore preferred. Binding pairs with dissociation velocity constants exceeding 0.01 per second are more preferred, and binding pairs with dissociation velocity constants according 0.02 per second even more preferred.

If kinetic reading is applied, i.e. measuring the change in the fluorescence signal per time unit or within a defined time interval, measurements can be made before a new equilibrium is established, and still the signal change can be used to calculate the analyte concentration or analyte concentrations. Thus, in the competitive embodiment of the present invention kinetic readings therefore allow the use binding pairs with dissociation velocity constants that are considerably lower than those used for endpoint readings. Then binding pair with dissociation velocity constants as low as 0.0001 per second can be used, but more preferred are dissociation velocity constants exceeding 0.001 per second, and still more preferred are dissociation velocity constants exceeding 0.005 per second.

A special embodiment of the method according to the present invention is to use a reagent in accordance with the present invention, and measure the change in degree of polarization of the fluorescence signal per time unit, or as a function of time, or within a given time interval after mixing in the sample material. In this embodiment the fluorescence signal or fluorescence signals can be read as a function of time, either as a continuous reading within a period of time, or as change per time unit between 2 or more points of time, or as an absolute change between 2 or more points of time. Such measurements are often called kinetic measurements or readings. The method according to present invention can also use kinetic reading methods with the other forms of fluorescence measurement methods described. The above mentioned applies to both the competitive and the non-competitive embodiments of the method according to the present invention.

An applicable embodiment of the present invention is further to use the above mentioned kinetic fluorescence polarization measurement, combined with the use of a reagent in accordance with the present invention, for which the wavelength of the maximum absorption coefficient for the fluorescent residues is higher than 600 nm or further preferred 620 nm or even more preferred exceeding 640 nm. For analysis of samples comprising hemoglobin, the maximum absorption coefficient should exceed 620 nm, since the hemoglobin interferes substantially with the concentration determination of the analyte or analytes. Such interference have usually been a major problem when fluorescence polarisation assay has been used with e.g. blood.

In the non-competitive embodiment of the present invention, the fluorescent residues will usually, but not always, be bound to the specific binding molecule. The specific binding molecule may be present in excess compared to the test sample/analytes molecules to be added, and the fluorescence signal that may be generated will change if analyte molecules have bound to the specific binding molecules. A good example of such a suitable specific binding molecule is aptamers, described in <<Selection of singlestranded DNA molecules that bind and inhibit human thrombin>>, by Bock & al., Nature vol. 355 pp 564-56, 1992. This article refers to a generic technology as general basis for production of specific binding molecules, and shows that aptamers comprising the nucleotide sequence GGTTGGTGTGGTTGG or GGTTGG are specifically bound to human thrombin.

Aptamers can also be used in the competitive embodiments of this invention, as well as in the non-competitive embodiments, Gold & al. in <<Diversity of oligonucleotide functions>> in Annel. Rev. Bichem. vol 64, pp 763-97, 1995, describe a large number of examples of use of the generic aptamer technology for production of specific binders, that can also be used in the reagent according to the present invention. The specific binder that is assigned to theophyllins is an example that is especially well suited for the competitive embodiments, whereas the RNA-aptamer described in the same place, which binds T4 DNA polymerase, is suited for the non-competitive embodiments.

Rick Konrad et al. in <<Isoenzyme-specific inhibition of Protein Kinase by RNA aptamers>> in J. Biol. Chem. vol. 269, pp 32051-54, 1994, have described two different aptamer frequencies suitable as specific binding molecules for Protein Kinase C, which may serve an Example of specific binding molecules to be used in the reagent according to the present invention.

Both in the competitive and the non-competitive embodiments of the present invention, specific binding molecules that include peptide sequences may be used. An example of a peptide sequence that is especially suitable in the non-competitive embodiment is the peptide sequence described by Chakravarthy & al. in Anal. Biochem. vol. 196, 144-150, 1991. Other examples of peptide sequences usefull as specific binders are described by Yue et al. in The Journal of Biological Chemistry, vol 271, p. 22245-22250, 1996. They were able to identify peptides that bind C-reactive protein in a dot blot assay employing numerous reagents, radioactive substances and autoradiography, a time consuming and expensive procedure.

The reagent in accordance with the invention, and in both the competitive and the non-competitive embodiment of the method, may be characterized by the fact that it is prepared solely for concentration determination of an analyte. The reagent may nevertheless, if so desired, comprise more than one specific binding molecule, and if desired, more than one specific binding molecule may include said fluorescent substance or include several fluorescent substances. The reagent may however also be characterized by the fact that it is prepared for concentration determination of several analytes, if desired—simultaneously, and—if desired—with several different specific binding molecules for each analyte. If so desired it is possible to use more than one of these binding pairs which include a binding partner with a fluorescent residue, or even several different fluorescent residues bound to different binding partners in different binding pairs. An almost unlimited amount of possibilities for signal-providing systems exists here. Several signal-providing systems will potentially increase the precision of the concentration determinations, but will at the same time increase the complexity of the measurement systems.

Traditional fluorescent substances such as fluorescein and Texas Red, Oregon Green, Rhodamin, tetramethyl-rhodamin, amino methyl coumarin, as well as a wide range of other substances may be used as fluorescent residues, and is well known in the prior art The prior art also provides method description for binding of the fluorescent substances to amino groups, thiol groups, alcohol groups, ketones, dioles and carboxylic acids. Furthermore it provides methods for binding of fluorescent substances to peptides, proteins, antibodies, nucleic acid polymers such as aptamers and other polymers which, however, is well known for a person skilled in the art.

Often, substantial amounts of hemoglobin or bilious pigments are present in the sample material during medical and biological use of the present invention. Then reagent comprising fluorescent substances with excitation or emission wavelengths that are absorbed by hemoglobin or bilirubin will often be influenced by the light absorption that characterizes the said substances. As already mentioned, an especially preferred embodiment of the present invention when analysing whole blood or blood lysates, is therefore based on reagents with fluorescent molecules for which the wavelength for maximum absorption coefficient of the fluorescent residues exceeds 600 nm, or more preferred 620 nm or even more preferred exceeding 640 nm. For analysis of samples comprising hemoglobin, the maximum absorption coefficient should exceed 620 nm since the hemoglobin interferes substantially with the concentration determination of the analyte or analytes. Such substances are sold by the company Amersham Pharmacia Biotech. under labels such as CyDye FluoroLink Reactive Dyes, with varying excitation wavelength (varying with analogues of the chemical structure), and with varying numbers of activated groups for binding to the substance that is to be labeled. More binding points will typically reduce the molecule's degree of freedom of rotation, and reduce the possibility for using fluorescence polarization measurement methods.

Suitable cyanine dyes are further described in U.S. Pat. No. 5,627,027: <<Cyanine dyes as labeling reagents for detection of biological and other materials by luminescence methods>> by Waggoner; Alan S, May 6, 1997. Furthermore, suitable substances are described in the prior art, such as e.g. in Waggoner et al U.S. Pat. No. 6,008,373 or Brush and Reimer U.S. Pat. No. 5,988,086 or Krandikar & al. U.S. Pat. No. 5,852,191 or Kusakata & al. U.S. Pat. No. 4,847,385 or Waggoner's U.S. Pat. No. 5,569,537.

The prior art also provides instructions for using the fluorescence substances group Biodypi, which provides good fluorescence signals exceeding 600, 620 and 640 nm, respectively. At present the substances Biodypi 630/650-X/MeOH, Biodypi 650/655-X/MeOH, Biodypi FL/MeOH, Biodypi R6G/MeOR Biodypi TMF-X/MeOH and Biodypi TR-X/MeOH, as well as the Alexa Floor Substances, are sold from the company Molecular Probes, and more similar substances can be expected. These substances are good alternatives to the cyanin dyes, and are among the preferred reagents in accordance with the present invention.

Suitable fluorescence residues can also have bound to them complex formers such as DTPA and N1 comprising complex-bound Lanthanide elements such as Europium, Samarium or Terbium. Advantageous qualities in these fluorescence residues are that they have very long Stokes-shifts (large difference in wavelength between excitation wavelength and emission wavelength), as well as a relatively long time interval between excitation and emission, which is preferred when using time dissolution fluorescence measurements. These fluorescent residues are well explained in Perkin Elmer Life Sciences' product catalogues.

Correspondingly the reagent in accordance with the present invention may be characterized by the fact that the fluorescent signal-providing substance partially consists of a polypeptide chain, and that this polypeptide chain preferably comprises less than 30 amino acids, and more preferred less than 20 amino acids, and even more preferred less than 12 amino acids.

Reffering to examples 17 and 18, where use of intermittent exitation at different wavelenghts is described for the analysis of several analytes using different exultation wavelenghts, it is preferred to use intermittent light with different wavelenghts, e.g. by the intermittent use of different diode lasers with different wavelength. It should be noted that the break between use of the different light sources should at least correspond to the lifetime for the exited condition of the actual fluorophore.

Best mode

Best mode is illustrated by the examples, preferably example 9 for the competitive embodiment of the invention, and by example 14 for the non-competitive embodiment of the invention.

### EXAMPLES

### Example 1

Fluorescent Binding Ligand for Whole Blood Analysis.

Synthesise the peptide Tyr-Trp-Ala-Asn-Phe-Ala-Arg-Asn-Arg-Asn by conventional technique, and dissolve it in pure water to a concentration of 2 mg per ml. Mix 50 ul of the said peptide solution with 50 ul sodium bicarbonate buffer pH=9.2. Mix 0.1 micromol Cy5 Fluorolink activated cyanin dye, supplied from Amerham Pharmacia Biotech, optionally dissolved in water-free DMSO with said solution and leav it to stand overnight at room temperature in darkness, and thereafter store the solution in a refrigerator in darkness. If wanted, 5 ul of 10 mmolar ethanolamine solution can be added to block any remaining activated fluorescent dye, or the solution—hereinafter called the stock solution—can be left to hydrolyse the non-reacted dye during storage.

Isolate pure Cy5-labelled Tyr-Trp-Ala-Asn-Phe-Ala-Arg-Asn-Arg-Asn from the stock solution by reverse phase chromatography in a C4 column (sold from many suppliers, including Waters, U.S.), using 0.1% trifluoroacetic acid as eluant and employ a gradient of 0% to 60% acetonitrile in 0.1% trifluoroacetic acid. Employ a photodetector coupled to a flow cell to monitor content of peptides by transmission measurements at 340 nm and Cy-5 by transmisson measurements at 650 nm, and isolate the Cy-5-conjugated peptide. Remove the trifluoroacetic acid and the acetonitrile and the water by lyophilisation.

Pure Cy5-labelled Tyr-Trp-Ala-Asn-Phe-Ala-Arg-Arg-Asn of the stock solution can also be isolated by thin layer chromatography. Apply aliquots of stock solution on silica gel plates and elute with n-butanol:acetic acid:water mixtures. Depending on the quality of the silica gel, the relative content of n-butanol:acetic acid:water can be adjusted to obtain ideal separation. After elution by conventional technique, dry the silica gel plate and inspect visually and by UV lamp (and optionally up nihydrin spray in parallell experiments) to identify the Cy5-labelled Tyr-Trp-Ala-Asn-Phe-Ala-Arg-Asn-Arg-Asn spot, separated from non-labelled peptide and free Cy-5 dye molecules. Isolate the silicagel comprising Cy5-labelled Tyr-Trp-Asn-Phe-Ala-Arg-Asn-Arg-Asn by scissors or spatulum. Suspend the isolatedsilica gel in 10 mM TRIS-buffer pH=8.0, whereby the Cy5-labelled Tyr-Trp-Ala-Asn-Phe-Ala-Arg-Asn-Arg-Asn is eluted into solution. The silica gel settles in the bottom of the tube. Decant off the TRIS-buffered solution with the purified Cy5-labelled Tyr-Trp-Ala-Asn-Phe-Ala-Arg-Asn-Arg-Asn.

If wanted, and for up-scaling, other conventional HPLC separation techniques well known to the skilled man of the art can be used in stead of thin layer chromatography.

### Example 2

Fluorescent Binding Ligand for Whole Blood Analysis.

Perform the method of the present invention according to example 1, except use Tyr-Trp-Ala-Asn-Phe-Ala-Arg-Asn-Gly-Asn instead of Tyr-Trp-Ala-Asn-Phe-Ala-Arg-Asn-Arg-Asn, to obtain Cy5-labelled Tyr-Trp-Ala-Asn-Phe-Ala-Arg-Asn-Gly-Asn.

### Eksempel 3

Method for Measurement of C-reactive Protein in Samples of Whole Blood.

Make an assay reagent by making a buffer of 150 mM sodium chloride 100 mM phosphate, pH=7.4. To this buffer, add bovine gammaglobulin from Sigma to a concentration of 2 mg/mL, and Triton X-100 from Pierce Chemical Company, US, to a final concentration of 0.1% v/v. Reagents of high purity with very low background fluorescence should be chosen. Add 1.0*10ER−11 mol per ml of Cy5-labelled Tyr-Trp-Ala-Asn-Phe-Ala-Arg-Asn-Arg-Asn according to example 1 above, and optionally add a suitable bacterostatic agentlike 0.01% sodium azide for prolonged storage. Keep aliquots of this mixture, e.g. 1 ml, in separate containers to be combined with unknown blood samples or aliquots of blood samples.

At the time for the determination of the C-reactive protein, take an aliquot, e.g. a volume of 20 ul of the whole blood sample e.g. by the use of a pre-measured capillary, and combine thereater the content of this pre-measured capillary with the assay reagent in the said separate container, the said separate container being kept in a place where the temperature is regulated to 32 degrees of Celcius.

Typically, use a container which is in the form of a cuvette with 4 polished transparent sides to be measured in an instrument built for fluorescent polarisation measurements, but said container comprising a removable stopper or seal which allows the capillary to enter the container, either by simply dropping the capillary or by introducing it through the seal. Furthermore, in its preferred that the capillary/container is designed so that the capillary falls to the bottom of the container and does not interfere with the excitation light or the emission light (see below). Thereafter, shake the container, and the blood flows out of the capillary and the cells are being lysed by the assay reagent. The C-reactive protein of the test sample aliquot starts to react with the Cy5-labelled Tyr-Trp-Ala-Asn-Phe-Arg-Asn-Arg-Asn of the assay reagent.

Immediately after the shaking, place the container in the fluorescence polarisation measurement instrument, having a temperature of 32 degrees of Celcius. For measurement of polarisation of the emitted fluorescence, the mixture in the container is irradiated with polarized light of 650 nm wavelength, typically by the use of a small polarized laser diode in the instrument, and when the polarisation of the emitted light constant, measure its polarisation a wavelength of 670 nm, with a rather narrow bandwidth, if the instruments allows for that. Calculate the concentration of C-reactive protein of the unknown sample by interpolation of the polarisation value of the emitted light measured on a standard curve obtained by measurement of standards of known C-reactive protein concentration. Such a standard curve can often be stored on the computer of the measurement instrument, enabling a direct reading of the concentration of C-reactive protein on the instrument.

If a measurement instrument with a flow cell is used, then bring the mixture to pass through the flow cell for measurement, according to the instructions given by the instrument manufacturer.

Typically, with a mild bacterial infection, the content of C-reactive protein of a 20 μl sample is between 10 and 100 mg per 1, but event much higher values can be seen in severe clinical conditions. On the other side, especially in screening of risk for heart disease and low grade of inflammation, measurement of values below 1 mg per liter is of interest. Furthermore, each C-reactive protein molecule can react with five molecules of Cy5-labelled Tyr-Trp-Ala-Asn-Phe-Ala-Arg-Asn-Arg-Asn. The concentrations of interest for C-reactive protein therefore varies a lot, and the concentration of Cy5-labelled Tyr-Trp-Ala-Asn-Phe-Ala-Arg-Asn-Arg-Asn therefore may have to be adjusted compared to the blood volume to be combined with the assay reagent.

### Example 4

Method for Measurement of C-reactive Protein in Samples of Whole Blood.

Perform the method of the present invention according to example 3, except use Cy5-labelled Tyr-Trp-Ala-Asn-Phe-Ala-Arg-Asn-Gly-Asn in the place of Cy5-labelled Tyr-Trp-Ala-Asn-Phe-Ala-Arg-Asn-Arg-Asn. This example of the method is more adequate for very high concentrations of C-reactive protein than example 3 because of Cy5-labelled Tyr-Trp-Ala-Asn-Phe-Ala-Arg-Asn-Gly-Asn lower affinity for C-reactive protein. Correspondingly, this example of the method is less appropriate to use for low concentrations of C-reactive protein.

### Example 5

Method for Measurement of C-reactive Protein in Samples of Whole Blood.

Perform the method of the present invention according to example 3, except however, instead of reading the polarisation after the polarisation value has become stable, measure the polarisation value as a function of time in a kinetic manner. If available, connect the measurement instrument to a computer recording the signal continuously or at different time points, optionally defined by a software. Compare then the recorded values to values obtained by measurement of standards of known C-reactive protein concentration as a function of time. Optionally, record a three-dimensional standard curve with polarisation values, different times and C-reactive protein concentration values, and compare the values obtained with the unknown sample to calculate the C-reactive protein concentration, optionally by the use of the least-square methods for best fit according to standard textbooks of statistics, optionally by means of a computer or another artificial memory connected to the measurement instrument.

### Example 6

Method for Measurement of C-reactive Protein in Samples of Whole Blood.

Perform the method of the present invention according to example 5, except keep the said separate containers (to be combined with unknown blood samples or aliquots of blood samples) at the room temperature, and use a electronic temperature measurement device in or at the fluorescence polarisation instrument, and use a four-dimensional standard curve, the forth dimension being temperature, in addition to polarisation values, different times and C-reactive protein concentration values, and compare the values obtained with the unknown sample to calculate the C-reactive protein concentration, optionally by the use of the least-square methods for best fit according to standard textbooks of statistics, optionally by means of a computer or another artificial memory connected to the measurement instrument.

### Example 7

Synthesis of Cynanin-5 Analogue of Theophyllin.

Make a synthesis of 8-(3-carboxypropyl)-1,3-dimethylxanthin anhydrid as described in Research Communications in Chemical Pathology and Pharmacology, vol. 13, p. 497-505, 1976. Dissolve diaminopropanol in water-free tetrahydrofuran. In another flask, dissolve half of the equimolar amount of the said 8-(3-carboxypropyl)-1,3-dimethylxanthin anhydrid in water-free tetahydrofuran. Add the said 8-(3-carboxypropyl)-1,3-dimethylxanthin anhydrid solution drop-wise to the diaminopropanol solution while stirring, and let the resulting solution react over night at room temperature. Optionally purify the resulting adduct by HPLC chromatography using conventional techniques well known to the skilled man of the art, if less consumption of activated cyanin dye is wanted (see below).

Thereafter, dissolve 6 times the molar amount which was used for diaminopropanol, of Cy5 Fluorolink activated cyanin dye supplied from Amersham Pharmacia Biotech, U.K., in water-free tetrahydrofuran, and add it previously described solution while stirring. Leave the resulting mixture to react over night a room temperature in darkness. In this way, a stock solution of non-pure 8-(3-carboxypropyl)-1,3-dimethylxanthin adduct with Cy5 Fluorolink activated cyanin dye with a water-soluble diaminopropanol spacer is obtained.

Purify the resulting 8-(3-carboxypropyl)-1,3-dimethylxanthin adduct with Cy5 Fluorolink activated cyanin dye with a water-soluble diaminopropanol spacer by means of thin layer chromatography according to example 1, and adjust the volumes of n-butanol, acetic acid and water in the elution mixture depending on the quality of the silica gel plates to obtain good separation. After elution by conventional technique, dry the silica gel plate and inspect visually and by UV lamp to identify the spot of 8-(3-carboxypropyl)-1,3-dimethylxanthin adduct with Cy5 Fluorolink spot. Isolate the silicagel 8-(3-carboxypropyl)-1,3-dimethylxanthin adduct with Cy5 Fluorolink by scissors or spatulum. Suspend the isolated silica gel in 10 mM TRIS-buffer pH=8.0, whereby 8-(3-carboxypropyl)-1,3-dimethylxanthin adduct with Cy5 Fluorolink is eluted into solution. The silica gel settles in the bottom of the tube. Decant off the TRIS-buffered solution with the purified 8-(3-carboxypropyl)-1,3-dimethylxanthin adduct with Cy5 Fluorolink.

If wanted, and for upscaling, conventional HPLC separation techniques well known to the skilled man of the art can be used in stead of thin layer chromatography. E.g Use the HPLC method described in example 1, however using a C4 or a C6 reversed phase column.

### Exampel 8

Fab-fragments of Antibodies with Affinity for Theophyllin.

Synthesis 8-(3-carboxypropyl)-1,3-dimethylxanthin anhydrid and prepare an albumin conjugate antigen as described in Research Communications in Chemical Pathology and Pharmacology, vol. 13, p. 497-505, 1976. Using this antigen, make mouse hybridomas. Select a clone the is specifically reactive to the Cyanin-5-analogue of theophyllin in example 7. The prior art teaches several ways of screening hybrodimas in textbooks on monoclonal antibody technology. The inventor of the present invention, however, prefers to make 150 mM sodium chloride 100 mM phosphate, pH=7.4. To this buffer, add bovine gammaglobulin from Sigma to a concentration of 2 mg/ml, and Triton X-100 from Pierce Chemical Company, US, to a final concentration of 0.1% v/v. Reagent is of high purity with very low background fluorescence should be chosen.

Then add Cynanin-5 analogue of theophyllin, made according to example 7, to a final concentration of 2 mg per l. Measure the polarisation of the emitted fluorescence when the mixture in the container is irradiated with polarized light of 650 nm wavelength, typically by the use of a small polarized laser diode in the instrument, and when the polarisation of the emitted light constant, measure its polarisation a wavelength of 670 nm, with a rather narrow bandwidth, if the instruments allows for that. Under monitoration by such fluorescence polarisation measurements, add antibodies isolated from the hybridoma cells to be investigated. For each addition, wait until the fluorescence polarisation signal is stable, before adding more antibody. Select antibodies from one or more hybridoma which specifically increases the fluorescence polarisation of the mixture, and do not select (often called counterselect) antibodies from hybridomas whose effect on the fluorescence polarisation technology is blocked or very significantly reduced by components of hums serum or serum from human individuals not treated with theophyllin. Especially check that the antibodies isolated from the hybridoma in question do not react with cafein or other drugs or food constituents that resembels theophyllin.

When antibodies from the selected hybridoma has been isolated, prepare FAB fragments by the use the ImmnuPure Fab preparation kit supplied by Pirece Chemical Company, and follow the instruction for the said kit.

### Example 9

Method to Determine the Concentration of Theophyllin in Whole Blood by the Use of a Fluorescent Immunocomplex.

Make anti-theophyllin FAB figments of antibodies according to example 8 and a Cynanin-5 analogue of theophyllin according to example 7.

Thereafter, make a tentative assay reagent at 32 degrees Celcius according to the following protocol: Make 150 mM sodium chloride 100 mM phosphate, pH=7.4. To this buffer, add bovine gammaglobulin from Sigma to a concentration of 2 mg/ml, and Triton X-100 from Pierce Chemical Company, US, to a final concentration of 0.1% v/v. Reagents of high purity with very low background fluorescence should be chosen.

Then add Cynanin-5 analogue of theophyllin, made according to example 7, to a final concentration of 2 mg per l. Measure the polarisation of the omitted fluorescence when the mixture in the container is irradiated with polarized light of 650 nm wavelength, typically by the use of a small polarized laser diode in the instrument, and when the polarisation of the emitted light constant, measure its polarisation a wavelength of 670 mm, with a rather narrow bandwidth, if the instruments allows for that. Under monitoration by such fluorescence polarisation measurements, add said anti-theophyllin FAB fragments. For each addition, wait until the fluorescence polarisation signal is stable, before adding more FAB fragments. Continue the addition of FAB fragments until noe more significant increase of polarisation value is measured with increased concentration of FAB fragments. Record this value of fluorescence polarisation. Subtract the initial fluorescence polarisation value, and calculate the difference between the values, hereinafter called <<polarisation value difference>>.

Start over again and make a final assay reagent identical to the tentative assay reagent, however, end the addition of said FAB fragments when the fluorescence polarisation value is equal to the initial polarisation value+80% of the <<polarisation value difference>>.

The final assay reagent is now ready, and optionally add a suitable bacterostatic agentlike 0.01% sodium azide for prolonged storage. Keep aliquots of this mixture, e.g. 1 ml, in separate containers to be combined with unknown blood samples or aliquots of blood samples.

At the time for the determination of concentration of theophyllin in blood samples, take an aliquot, e.g. a volume of 20 μl of the whole blood sample e.g. by the use of a pre-measured capillary, and combine thereafter the content of this pre-measured capillary with the assay reagent in the said separate container, the said separate container bring kept in a place where the temperature is regulated to 32 degrees of Celcius. Typically, use a container as described in example 3. Thereafter, shake the container, and the blood flows out of the capillary and the cells are being lysed by the assay reagent theophyllin of the test sample aliquot starts to displace the fluorescent theophyllin of the immunocomplexes of the assay reagent.

Immediately after the shaking, place the container in the fluorescence polarisation measurement instrument having a temperature of 32 degrees of Celcius. For measurement of polarisation of the emitted fluorescence, the mixture in the container is irradiated with polarized light of 650 nm wavelength, typically by the use of a small polarized laser diode in the instrument, and when the polarisation of the emitted light constant, measure its polarisation a wavelength of 670 nm, with a rather narrow bandwidth, if the instruments allows for that. Calculate the concentration of theophyllin of the unknown sample by interpolation of the polarisation value of the emitted light measured on a standard curve obtained by measurement of standards of known theophyllin concentration. Such a standard curve can often be stored on the computer of the measurement instrument, enabling a direct reading of the concentration of theophyllin on the instrument.

If a measurement instrument with a flow cell is used, then bring the mixture to pass through the flow cell for measurement, according to the instructions given by the instrument manufacturer.

If the measurement instrument allows it, much better precision is obtained if—instead of reading the polarisation after the polarisation value has become stable—the polarisation value as a function of time is measured in a kinetic manner. If available, connect the measurement instrument to a computer recording the signal continuously or at different time points, optionally defined by a software. Compare then the recorded values to values obtained by measurement of standards of known theophyllin concentration as a function of time. Optionally, record a three-dimensional standard curve with polarization values, different times and theophyllin concentration values, and compare the values obtained with the unknown sample to calculate the theophyllin concentration, optionally by the use of the least-square methods for best fit according to standard textbooks of statistics, optionally by means of a computer or another artificial memory connected to the measurement instrument. Furthermore, if you have access to a temperature recording instrument keep the said separate containers (to be combined with unknown blood samples or aliquots of blood samples) at the room temperature, and use the temperature measurement device in or at the fluorescence polarisation instrument, and use a four-dimensional standard curve, the forth dimension being temperature, in addition to polarisation values, different times and theophyllin concentration values, and compare the values obtained with the unknown sample to calculate the theophyllin concentration, optionally by the use of the least-square methods for best fit according to standard textbooks of statistics, optionally by means of a computer or another artificial memory connected to the measurement instrument.

The concentration of theophyllin in blood samples varies significantly. The main interest is measurements of therapeutic concentration values. However, higher and toxic values are of interest in forensic medicine and lower concentrations are of interests in sports medicine. In the performance of this example of the method of the present invention, please therefore adjust the concentration of the Cynanin-5 analogue of theophyllin, and hence the FAB fragment concentration. At high concentration ranges, FAB fragments of high dissociation rate constant must be chosen. At low concentration ranges, use FAB fragments with both high association rate constant and high dissociation rate constant.

### Example 10

Fluorescent Peptide for Determination or Concentration of Albumin in Urine.

Synthesise the peptide Asp-Ala-His-Lys-Ser-Glu-Val-Ala (the N-terminal peptide of human albumin) by conventional technique, and dissolve it in pure water to a concentration of 2 mg per ml. Mix 50 μl of the said peptide solution with 50 ul sodium bicarbonate buffer pH=9.2. Mix 0.1 micromol 6-carboxyfluorescein-N-hydroxysuccinimide, optionally dissolved in water-free DMSO with said solution and leave it to stand overnight at room temperature in darkness, and thereafter store the solution in a refrigerator in darkness. If wanted, 5 μl of 10 mmolar ethanolamine solution can be added to block any remaining activated fluorescent dye, or the solution—hereinafter called the stock solution—can be left to hydrolyse the un-reacted dye during storage.

Purify fluorescein labelled peptide from the stock solution by thin layer chromatography: Apply aliquots of stock solution on silica gel plates and elute with n-butanol:acetic acid:water in a mixture. Depending on the quality of the silica gel, the relative content of n-butanol:acetic acid:water can be adjusted to obtain ideal separation. After elution by conventional technique, dry the silica gel plate and inspect visually and by UV lamp (and optionally using nihydrin spray in parallell experiments) to identify the fluorescein labelled peptide spot, separated from non-labelled peptide and free fluorescein dye molecules. Isolate the silicagel comprising fluorescein labelled peptide by scissors or spatulum. Suspend the isolatedsilica gel in 50 mM BIS-TRIS-buffer pH=8.0, whereby fluorescein labelled peptide is eluted into solution. The silica gel settles in the bottom of the tube. Decant off the TRIS-buffered solution with the purified fluorescein labelled peptide.

If wanted, and for upscaling, conventional HPLC separation techniques well known to the skilled man of the art can be used in stead of thin layer chromatography. E.g. Use the HPLC method described in example 1.

### Example 11

Method to Determine the Concentration of Albumin in Urine by the Use of a Fluorescent Immunocomplex

Make anti-Asp-Ala-His-Lys-Ser-Glu-Val-Ala antibodies using conventional techniques well known to the skilled man of the art, e.g. by the use of an antigen formed by coupling of the peptide to keyholde limpet cyanin. Alternatively, purchase the peptide synthesis and the raising of antibodies from a service providing company, e.g. by Eurogenentech of Belgium. Make FAB fragments from the antibodies antibodies by the use the ImmnuPure Fab preparation kit supplied by Pirece Chemical Company, and follow the instruction for the said kit.

Make fluorescein labelled peptide as described in example 10. On selection of monoclonal antibody, see below.

Thereafter, make a tentative assay reagent at 32 degrees Celcius according to the following protocol: Make 150 mM sodium chloride 100 mM phosphate, pH=7.4. To this buffer, add bovine gammaglobulin from Sigma to a concentration of 2 mg/ml, and Triton X-100 from Pierce Chemical Company, US, to a final concentration of 0.01% v/v. If necessary to remove protein reactive to the monoclonal antibody to be used (see below), pass the solution over an immunosorbent with anti-human albumin monoclonal antibodies, by the use of conventional immunoadsorbtion techniques well known to the skilled man of the art.

Reagents of high purity with very low background fluorescence should be chosen.

Then add said fluorescein labelled peptide to a final concentration 1.0*10E−9 mol per 1. Measure the polarisation of the emitted fluorescence when the mixture in the container is irradiated with polarized light of 475 nm wavelength, and when the polarisation of the omitted light constant, measure its polarisation a wavelength of 525 nm, with a rather narrow bandwidth, typical of 10 nm, if the instruments allows for that. Under monitoration by such fluorescence polarisation measurements, add said anti-albumin FAB fragments. For each addition wait until the fluorescence polarisation signal is stable, before adding more FAB fragments. Continue the addition of FAB fragments until no more significant increase of polarisation value is measured with increased concentration of FAB fragments. Record this value of fluorescence polarisation. Subtract the initial fluorescence polarisation value, and calculate the difference between the values, hereinafter called <<polarisation value difference>>.

Start over again and make a final assay reagent identical to the tentative assay reagent, however, end the addition of said FAB anti-albumin fragments when the fluorescence polarisation value is equal to the initial polarisation value+80% of the <<polarisation value difference>>.

The final assay reagent is now ready, and optionally add a suitable bacterostatic agent like 0.01% sodium azide for prolonged storage. Keep aliquots of this mixture, e.g. 2 ml, in separate containers to be combined with unknown urine samples or aliquots of urine samples.

At the time for the determination of concentration of albumin in a urine samples, take an aliquot, e.g. a volume of 20 ul of the urine sample e.g. by the use of a pre-measured capillary, and combine thereafter the content of this pre-measured capillary with the assay reagent in the said separate container the said separate container being kept in a place where the temperature is regulated to 32 degrees of Celcius. Typically, use a container as described in example 3. Thereafter, shake the container, and the urine flows out of the capillary. Albumin of the test sample aliquot starts to displace the fluorescein labelled peptide of the immuno-complexes of the assay reagent.

Immediately after the shaking, place the container in the fluorescence polarization measurement instrument, having a temperature of 32 degrees of Celcius. For measurement of polarisation of the emitted fluorescence, the mixture in the container is irradiated with polarized light of 475 nm wavelength, and when the polarisation of the emitted light constant, measure its polarisation a wavelength of 525 nm, with a rather narrow bandwidth, typically 10 nm, if the instruments allows for that. Calculate the concentration of human albumin of the unknown sample by interpolation of the polarisation value of the emitted light measured on a standard curve obtained by measurement of standards of known human albumin concentration. Such a standard curve can often be stored on the computer of the measurement instrument, enabling a direct reading of the concentration of albumin.

If a measurement instrument with a flow cell is used, then bring the mixture to pass through the flow cell for measurement, according to the instructions given by the instrument manufacturer.

If the measurement instrument allows it, much better precision is obtained if—instead of reading the polarisation after the polarisation value has become stable—the polarisation value as a function of time is measured in a kinetic manner. If available, connect the measurement instrument to a computer recording the signal continuously or at different time points, optionally defined by a software. Compare then the recorded values to values obtained by measurement of standards of known human albumin concentration as a function of time. Optionally, record a three-dimensional standard curve with polarisation values, different times and human albumin concentration values, and compare the values obtained with the unknown sample to calculate the human albumin concentration, optionally by the use of the least-square methods for best fit according to standard textbooks of statistics, optionally by means of a computer or another artificial memory connected to the measurement instrument. Furthermore, if you have access to a temperature recording instrument keep the said separate containers (to be combined with unknown blood samples or aliquots of blood samples) at the room temperature, and use the temperature measurement device in or at the fluorescence polarisation instrument, and use a four-dimensional standard curve, the forth dimension being temperature, in addition to polarisation values, different times and human albumin concentration values, and compare the values obtained with the unknown sample to calculate the human albumin concentration, optionally by the use of the least-square methods for best fit according to standard textbooks of statistics, optionally by means of a computer or another artificial memory connected to the measurement instrument.

The concentration of albumin in urine samples varies significantly. Reference values are in the range of up to 20 mg per liter. Slightly elevated values of uncertain origin are found in some healthy individuals, individuals who may stay healthy to old ages. Moderate elevation of the albumin values are seen as early signs of diabetic kidney damage. In severe kidney disease, urine albumin concentration may rise to many hundred mgs per liter. In the performance of this example of the method of the present invention, please therefore adjust the concentration of the fluorescein labelled peptide, and hence the FAB fragment concentration. At high concentration ranges, FAB fragments of high dissociation rate constant must be chosen. At low concentration ranges, use FAB fragments with both high association rate constant and high dissociation rate constant.

### Example 12

Method for Measurement of Tobramycin in Whole Blood.

Synthetic RNA molecules which bind tobramycin is synthesised according to the article <<RNA molecules that specifically and stoichiomterically bind amioglycoaide antibiotics with high affinities>> by Wang & al., published in <<Biochemistry>> 1996, 35, 12338-12346.

Buy Tobramycin from Fluka. Dissolve 200 umoles Tobramycin in 500 ul water. Add 500 ul dimethylformamide. Cool the solution to 5 degrees Celcius. Equimolar concentration Cy5 Fluoro Link molecules in DMF solution is added, and the mixture is stirred at 5 degrees Celcius for 2 hours.

Elute the mixture CG 50 cathion exchange chromatography, with a gradient of 0 to 500 mM ammonium hydroxyde.

The desired conjugated is thereby isolated and lyophilized to remove the ammonium hydroxyde.

Make an assay buffer consisting of 140 mM NaCl, 5 mM KCl, 1 mM manganes chloride. 1 mM calcium chloride and 20 mM Hepes, and 3 mg bovine gamma globulin per ml, and pH=7.4. Add 30 ng Tobramycin-Cy-5-conjugate per ml assay solution, and thereafter add an equimolare mount of the synthesised RNA molecules.

The final assay reagent is now ready, and optionally add a suitable bacterostatic agentlike 0.01% sodium azide for prolonged storage. Keep aliquots of this mixture, e.g. 1 ml, in separate containers to be combined with unknown blood samples or aliquots of blood samples.

At the time for the determination of concentration of Tobramycin in blood samples, take an aliquot, e.g. a volume of 20 μl of the blood sample sample e.g. by the use of a pre-measured capillary, and combine thereafter the content of this pre-measured capillary with the assay reagent in the said separate container, the said separate container being kept in a place where the temperature is regulated to 32 degrees of Celcius. Typically, use a container as described in example 3. Thereafter, shake the container, and the blood flows out of the capillary. Tobramycin of the test sample aliquot starts to displace the Cy-5 conjugated Tobramycin of the RNA/Cy-5-Tobramycin complex of the assay reagent.

Immediately after the shaking, place the container in the fluorescence polarisation measurement instrument, having a temperature of 32 degrees of Celcius. For measurement of polarisation of the emitted fluorescence, the mixture in the container is irradiated with polarized light of 649 nm wavelength, and when the polarisation of the emitted light constant, measure its polarisation a wavelength of 670 nm, with a rather narrow bandwidth, typically 10 nm, if the instruments allows for that. Calculate the concentration of Tobramycin of the unknown sample by interpolation of the polarisation value of the emitted light measured on a standard curve obtained by measurement of standards of known human albumin concentration. Such a standard curve can often be stored on the computer of the measurement instrument, enabling a direct reading of the concentration of albumin.

If a measurement instrument with a flow cell is used, then bring the mixture to pass through the flow cell for measurement, according to the instructions given by the instrument manufacturer.

If the measurement instrument allows it, much better precision is obtained if—instead of reading the polarization after the polarisation value has become stable—the polarisation value as a function of time is measured in a kinetic manner. If available, connect the measurement instrument to a computer recording the signal continuously or at different time points, optionally defined by a software. Compare then the recorded values to values obtained by measurement of standards of known Tobramycin concentration as a function of time. Optionally, record a three-dimensional standard curve with polarisation values, different times and Tobramycin concentration values, and compare the values obtained with the unknown sample to calculate the Tobramycin concentration, optionally by the use of the least-square methods for best fit according to standard textbooks of statistics, optionally by means of a computer or another artificial memory connected to the measurement instrument. Furthermore, if you have access to a temperature recording instrument keep the said separate containers (to be combined with known blood samples or aliquots of blood samples) at the room temperature, and use the temperature measurement device in or at the fluorescence polarisation instrument, and use a four-dimensional standard curve, the forth dimension being temperature, in addition to polarisation values, different times and Tobramycin concentration values, and compare the values obtained with the unknown sample to calculate the Tobramycin concentration, optionally by the use of the least-square methods for best fit according to standard textbooks of statistics, optionally by means of a computer or another artificial memory connected to the measurement instrument.

The concentration of Tobramycin in blood samples varies significantly, dependant on for what the assay is used. Measurements of therapeutic concentrations varies with clinical indications, and measurements of blood concentrations in pharmacokinetic studies will be different. In the performance of this example of the method of the present invention, please therefore adjust the concentration of the Tobramycin-5-conjugate and RNA molecules to the appropriate level. The J6RNA RNA molecule published in <<Biochemistry>> 1996, 35, 12338-12346, is appropriate at low concentrations, other RNA molecules with lower affinity can be identified by the Selex technology and by synthesis methods well known to the skilled man of the art.

### Example 13

Fluorescent Insulin for Determination of Concentration of Human Auto-anti-insulin Antibodies.

Synthesise human insulin by conventional technique, and dissolve it in pure water to a concentration of 2 mg per ml. Mix 50 ul of the said peptide solution with 50 ul sodium bicarbonate buffer pH=9.2. Mix 0.2 Cy5 Fluorolink activated cyanin dye, supplied from Amersham Pharmacia Biotech, optionally dissolved in water-free DMSO with said solution and leave it to stand overnight at room temperature in darkness, and thereafter store the solution in a refrigerator in darkness. If wanted, 5 ul of 10 mmolar ethanolamine solution can be added to block any remaining activated fluorescent dye, or the solution—hereinafter called the stock solution—can be left to hydrolyse the un-reacted dye during storage.

Purify Cy-5 labelled insulin from the stock solution by reverse phase chromatography in C4 column (sold from many suppliers, including Waters, U.S.), using 0.1% trifluoroacetic acid as eluant and employ a gradient of 0% to 60% acetonitrile in 0.1% trifluoroacetic acid. Employ a photodetector coupled to a flow cell to monitor content of peptides by transmission measurements at 340 nm and Cy-5 by transmisson measurements at 650 nm, and isolate the Cy-5-conjugated insulin. Remove the acetonotrile and the trifluoracetic acid by lyophilization.

Alternatively, other HPLC methods for isolation of peptide conjugutes well known to the skilled man of the art may be chosen.

### Eksempel 14

Method for Measurement of Anti-insulin Antibodies in Samples of Whole Blood.

Make an assay reagent by making a buffer of 150 mM sodium chloride 100 mM phosphate pH=7.4. To this buffer, add insulin free bovine gammaglobulin from Sigma to a concentration of 2 mg/ml and Triton X-100 from Pierce Chemical Company, US, to a final concentration of 0.1% v/v. Reagents of high purity with very low background fluorescence should be chosen. Add e.g. 1.0*10E−12 mol per ml (for choice of concentration, see below) of Cy5-labelled human insulin according to example 13 above, and optionally add a suitable bacterostatic agentlike 0.01% sodium azide for prolonged storage. Keep aliquots of this mixture, e.g. 1 ml, in separate containers to be combined with unknown blood samples or aliquots of blood samples.

At the time for the determination of concentration of anti-insulin antibodies in a blood sample, take an aliquot, e.g. a volume of 20 μl of the whole blood sample e.g. by the use of a pre-measured capillary, and combine thereafter the content of this pre-measured capillary with the assay reagent in the said separate container, the said separate container being kept in a place where the temperature is regulated to 32 degrees of Celcius. Typically, use a container is in the form of a cuvette with 4 polished transparent sides to be measured in an instrument built for fluorescent polarisation measurements, but said container comprising a removable stopper or seal which allows the capillary to enter the container, either by simply dropping the capillary or by introducing it through the seal. Furthermore, in its preferred that the capillary/container is designed so that the capillary falls to the bottom of the container and does not interfere with the excitation light or the emission light (see below). Thereafter, shake the container, and the blood flows out of the capillary and the cells are being lysed by the assay reagent. Anti-insulin antibodies of the test sample aliquot starts to react with the Cy5-labelled insulin of the assay reagent.

Immediately after the shaking, place the container in the fluorescence polarisation measurement instrument, having a temperature of 32 degrees of Celcius. For measurement of polarisation of the emitted fluorescence, the mixture in the container is irradiated with polarized light of 650 nm wavelength, typically by the use of a small polarized laser diode in the instrument, and when the polarisation of the emitted light constant, measure its polarisation a wavelength of 670 nm, with a rather narrow bandwidth, preferentially band withs of 5 nm. Calculate the concentration of insulin of the unknown sample by interpolation of the polarisation value of the emitted light measured on a standard curve obtained by measurement of standards of known insulin concentration. Such a standard curve can often be stored on the computer of the measurement instrument, enabling a direct reading of the concentration of insulin on the instrument.

If a measurement instrument with a flow cell is used, then bring the mixture to pass through the flow cell for measurement, according to the instructions given by the instrument manufacturer. Since insulin has a rather high molecular radius, a fluorescent polarisation instrument with a high precision in the measurement of the degree of polarisation of the light is prefered.

If the measurement instrument allows it, much better precision is obtained if—instead of reading the polarisation after the polarisation value has become stable—the polarisation value as a function of time is measured in a kinetic manner. If available, connect the measurement instrument to a computer recording the signal continuously or at different time points, optionally defined by a software. Compare then the recorded values to values obtained by measurement of standards of known anti-insulin antibody concentration as a function of time. Optionally, record a three-dimensional standard curve with polarisation values, different times and insulin antibody concentration values, and compare the values obtained with the unknown sample to calculate the concentration of anti-insulin antibodies, optionally by the use of the least-square methods for best fit according to standard textbooks of statistics, optionally by means of a computer or another artificial memory connected to the measurement instrument. Furthermore, if you have access to a temperature recording instrument keep the said separate containers (to be combined with unknown blood samples or aliquots of blood samples) at the room temperature, and use the temperature measurement device in or at the fluorescence polarisation instrument, and use a four-dimensional standard curve, the forth dimension being temperature, in addition to polarisation values, different times and anti-insulin antibody concentration values, and compare the values obtained with the unknown sample to calculate the anti-insulin antibody concentration, optionally by the use of the least-square methods for best fit according to standard textbooks of statistics, optionally by means of a computer or another artificial memory connected to the measurement instrument.

Anti-insulin antibodies are typically present in very low concentrations values in healthy subjects. Diabetics in mild or early phases of the condition will typically have low antibodies towards insulin, while patients having received insulin treatment for many years typically will have very high concentrations of antibodies towards insulin The concentration of the Cy-5-conjugated insulin and the total sample volume must therefore be chosen according to which kind of patients the sample is taken from. If the fluorescence polarisation measured is outside the standard curve obtained with the chosen concentration of Cy-5-insulin conjugate and sample volume, another concentration of Cy-5-insulin conjugate and sample volume must be chosen.

### Eksempel 15

Determination of Concentration in Urine by Means of a Ru-ligand Immunocomplex.

According to this example it is shown that it is possible to increase the molecular weight of the binding partner(s) with the fluorescent residue(s) attached, in order to determine the concentration of an analyte/analytes. However, as mentioned earlier, this requires the use of fluorescent residue(s) with longer decay time.

Make FAB-fragments from rabbit anti-human albumin antibodies, purchased from DakoAS, Denmark, using the ImmunoPure Fab preparation kit supplied by Pirece Chemical Company, and follow the instruction for the said kit.

Furthermore, make human serum albumin conjugate with Ru(bpy)dcbpy as described by Terpetsching & al. in Analytical Biochemistry 227, 140-147, 1995.

Thereafter, make a tentative assay reagent at 32 degrees Celcius according to the following protocol: Make 150 mM sodium chloride 100 mM phosphate, pH=7.4. To this buffer, add bovine gammaglobulin from Sigma to a concentration of 2 mg/ml and Triton X-100 from Pierce Chemical Company, US, to a final concentration of 0.01% v/v. Reagents of high purity with very low background fluorescence should be chosen.

Then add said human serum albumin conjugate with Ru(bpy)dcbpy to a final concentration 1.0*10E−9 mol per l. Measure the polarisation of the emitted fluorescence when the mixture in the container is irradiated with polarized light of 485 (alternatively 360, see the said article of Terpetsching) nm wavelength, and when the polarisation of the emitted light constant, measure its polarisation a wavelength of 660 nm, with a rather narrow bandwidth, typical of 10 nm, if the instruments allows for that. Under monitoration by such fluorescence polarisation measurements, add said anti-albumin FAB fragments. For each addition, wait until the fluorescence polarisation signal is stable, before adding more FAB fragments. Continue the addition of FAB fragments until no more significant increase of polarisation value is measured with increased concentration of FAB fragments. Record this value of fluorescence polarisation. Subtract the initial fluorescence polarisation value, and calculate the difference between the values, hereinafter called <<polarisation value difference>>.

Start over again and make a final assay reagent identical to the tentative assay reagent, however, end the addition of said FAB anti-albumin fragments when the fluorescence polarisation value is equal to the initial polarisation value+80% of the <<polarisation value difference>>.

The final assay reagent is now ready, end optionally add a suitable bacterostatic agent like 0.01% sodium azide for prolonged storage. Keep aliquots of this mixture, e.g. 2 ml, in separate containers to be combined with unknown urine samples or aliquots of urine samples.

At the time for the determination of concentration of albumin in a urine samples, take an aliquot, e.g. a volume of 20 μl of the urine sample e.g. by the use of a pre-measured capillary, and combine thereafter the content of this pre-measured capillary with the assay reagent in the said separate container, the said separate container being kept in a place where the temperature is regulated to 32 degrees of Celcius. Typically, use a container as described in example 3. Thereafter, shake the container, and the urine flows out of the capillary. Albumin of the test sample aliquot starts to displace the human serum albumin conjugate with Ru(bpy)dcbpy of the immuno-complexes of the assay reagent.

Immediately after the shaking, place the container in the fluorescence polarisation measurement instrument, having a temperature of 32 degrees of Celcius. For measurement of polarisation of the emitted fluorescence, see above in this example. Calculate the concentration of human albumin of the unknown sample by interpolation of the polarisation value of the emitted light measured on a standard curve obtained by measurement of standards of known human albumin concentration. Such a standard curve can often be stored on the computer of the measurement instrument, enabling a direct reading of the concentration of albumin.

If a measurement indent with a flow cell is used, then bring the mixture to pass through the flow cell for measurement, according to the instructions given by the instrument manufacturer.

If the measurement instrument allows it, much better precision is obtained if—instead of reading the polarisation after the polarisation value has become stable—the polarisation value as a function of time is measured in a kinetic manner. If available, connect the measurement instrument to a computer recording the signal continuously or at different time points, optionally defined by a software. Compare then the recorded values to values obtained by measurement of standards of known human albumin concentration as a function of time. Optionally, record a three-dimensional standard curve with polarisation values, different times and human albumin concentration values, and compare the values obtained with the unknown sample to calculate the human albumin concentration, optionally by the use of the least-square methods for best fit according to standard textbooks of statistics, optionally by means of a computer or another artificial memory connected to the measurement instrument. Furthermore, if you have access to a temperature recording instrument keep the said separate containers (to be combined with unknown blood samples or aliquots of blood samples) at the room temperature, and use the temperature measurement device in or at the fluorescence polarisation instrument, and use a four-dimensional standard curve, the forth dimension being temperature, in addition to polarisation values, different times and human albumin concentration values, and compare the values obtained with the unknown sample to calculate the human albumin concentration, optionally by the use of the least-square methods for best fit according to standard textbooks of statistics, optionally by means of a computer or another artificial memory connected to the measurement instrument.

The concentration of albumin in urine samples varies significantly. Reference values are in the range of up to 20 mg per liter. Slightly elevated values of uncertain origin are found in some healthy individuals, individuals who may stay healthy to old ages. Moderate elevation of the albumin values are seen as early signs of diabetic kidney damage. In severe kidney disease, urine albumin concentration may rise to many hundred mgs per liter. In the performance of this example of the method of the present invention, please therefore adjust the concentration of the human serum albumin conjugate with Ru(bpy)dcbpy, and hence the FAB fragment concentration. At high concentration ranges, FAB fragments of high dissociation rate constant must be chosen. At low concentration ranges, use FAB fragment with both high association rate constant and high dissociation rate constant.

### Example 16

Method for Measurement of Blood Theophyllin by the Use of an Aptamer Complex.

Synthesise the RNA aptamer which binds to theophyllin with an association affinity constant of 1.0*10E7/molar, described by R. D. Jenison & al in Science, vol 263, 1994.

Synthesise Cyanin-5 analouge of theophyllin according to example 7.

Make an assay buffer consisting of 140 mM NaCl, 5 mM KCl, 1 mM manganes chloride. 1 mM calcium chloride and 20 mM Hepes, and 3 mg bovine gamma globulin per ml, and pH=7.4. Add 2 mg Cyanin-5 analouge of theophyllin per 1 assay solution, and thereafter add an equimolare amount of the synthesised RNA molecules.

The final assay reagent is now ready, and optionally add a suitable bacterostatic agentlike 0.01% sodium azide for prolonged storage. Keep aliquots of this mixture, e.g. 1 ml, in separate containers to be combined with unknown blood samples or aliquots of blood samples.

At the time for the determination of concentration of theophyllin in blood samples, take an aliquot, e.g. a volume of 20 μl of the blood sample sample e.g. by the use of a pre-measured capillary, and combine thereafter the content of this pre-measured capillary with the assay reagent in the said separate container, the said separate container being kept in a place where the temperature is regulated to 32 degrees of Celcius. Typically, use a container as described in example 3. Thereafter, shake the container, and the blood flows out of the capillary. Theophyllin of the test sample aliquot starts to displace the Cyanin-5 analogue of theophyllin of the RNA/Cyanin-5 analogue of theophyllin complex of the assay reagent.

Immediately after the shaking, place the container in the fluorescence polarisation measurement instrument, having a temperature of 32 degrees of Celcius. For measurement of polarisation of the emitted fluorescence, the mixture in the container is irradiated with polarized light of 649 nm wavelength, and when the polarisation of the emitted light is constant, measure its polarisation at a wavelength of 670 nm, with a rather narrow bandwidth, typically 10 nm, if the instruments allows for that. Calculate the concentration of Theophyllin of the unknown sample by interpolation of the polarisation value of the emitted light measured on a standard curve obtained by measurement of standards of known human albumin concentration. Such a standard curve can often be stored on the computer of the measurement instrument, enabling a direct reading of the concentration of Theophyllin.

If a measurement instrument with a flow cell is used, then bring the mixture to pass through the flow cell for measurement, according to the instructions given by the instrument manufacturer.

If the measurement instrument allows it, a much bettor precision is obtained if—instead of reading the polarisation after the polarisation value has become stable—the polarisation value as a function of time is measured in a kinetic manner. If available, connect the measurement instrument to a computer recording the signal continuously or at different time points, optionally defined by a software. Compare then the recorded values to values obtained by measurement of standards of known theophyllin concentration as a function of time. Optionally, record a three-dimensional standard curve with polarisation values, different times and theophyllin concentration values, and compare the values obtained with the unknown sample to calculate the theophyllin concentration, optionally by the use of the least-square methods for best fit according to standard textbooks of statistics, optionally by means of a computer or another artificial memory connected to the measurement instrument. Furthermore, if you have access to a temperature recording instrument keep the said separate containers (to be combined with unknown blood samples or aliquots of blood samples) at the room temperature, and use the temperature measurement device in or at the fluorescence polarisation instrument, and use a four-dimensional standard curve, the forth dimension being temperature, in addition to polarisation values, different times and theophyllin concentration values, and compare the values obtained with the unknown sample to calculate the theophyllin concentration, optionally by the use of the least-square methods for best fit according to standard textbooks of statistics, optionally by means of a computer or another artificial memory connected to the measurement instrument.

The concentration of theophyllin in blood samples varies significantly. The main interest is measurements of therapeutic concentration values. However, higher and toxic values are of interest in forensic medicine, and lower concentrations are of interests in sports medicine. In the performance of this example of the method of the present invention, please therefore adjust the concentration of the Cynanin-5 analogue of theophyllin, and hence the RNA aptamer concentration.

### Example 17

Simultaneous Measurement of Human Choriongonadotropin and Albumin in Urine.

Purchase human beta-subunit choriongonadotropin (BHCG) from Sigma, and further purify it by size exclusion chromatography with mehods well known to the skilled man of the art, using 10 mM phosphate buffer pH=7.2 witm 0.15 M sodium chlorid as eluant. Label the human beta-subunit of choriongonadotropin with Cy-5 Fluorolink following the package insert of Amersham Pharmacia Biotech, and measure the content of Cy-5 per molecule BHCG according to the same method after purification by size exclusion chromatography, as recommended by Amersham Pharmacia Biotech.

Purchase monoclonal anti-BHCG from Chemicon Inc, US, and prepare FAB fragments from the antibodies according to the ImmunoPure FAB preparation kit from Pierce Cemical Company.

Make anti-Asp-Ala-His-Lys-Ser-Glu-Val-Ala antibodies using conventional techniques well known to the skilled man of the art, e.g. by the use of an antigen formed by coupling of the peptide to keyholde limpet cyanin. Alternatively, purchase the peptide synthesis and the raising of antibodies from a service providing company, e.g. by Eurogenentech of Belgium. Make FAB fragments from the antibodies antibodies by the use the ImmnuPure Fab preparation kit supplied by Pirece Chemical Company, and follow the instruction for the said kit.

Make fluorescein labelled peptide as described in example 10, and the reagent for determination of albumin in urine according to example 11. Thereafter, add to the said reagent for determination of urine albumin, Cy5-labelled BHCG 1/100 of molecular concentration of the BHCG level intended to measure in urine. (A suitable level to could be levels corresponding to 3000 U per l).

On selection of monoclonal antibody f, see below.

Measure the polarisation of the emitted fluorescence when the mixture in the container is irradiated with polarized light of 550 nm wavelength, and when the polarisation of the emitted light constants measure its polarisation a wavelength of 570 nm, with a rather narrow bandwidth, typical of 10 nm, if the instruments allows for that. Under monitoration by such fluorescence polarisation measurements, add said anti-BHCG FAB fragment. For each addition, wait until the fluorescence polarisation signal is stable, before adding more FAB fragments. Continue the addition of FAB fragments until no more significant increase of polarisation value is measured with increased concentration of FAB fragments. Record this value of fluorescence polarisation. Subtract the initial fluorescence polarisation value, and calculate the difference between the values, hereinafter called <<polarisation value difference>>.

Start over again and make a final assay reagent identical to the tentative assay reagent, however, end the addition of said FAB anti-BHCG fragments when the fluorescence polarisation value is equal to the initial polarisation value+80% of the <<polarisation value difference>>.

The final assay reagent is now ready, and optionally add a suitable bacterostatic agent like 0.01% sodium azide for prolonged storage. Keep aliquots of this mixture, e.g. 2 ml, in separate containers to be combined with unknown urine samples or aliquots of urine samples.

At the time for the determination of concentration of albumin and HCG in a urine samples, take an aliquot, e.g. a volume of 20 μl of the urine sample e.g. by the use of a pre-measured capillary, and combine thereafter the content of this pre-measured capillary with the assay reagent in the said separate container, the said separate container being kept in a place where the temperature is regulated to 32 degrees of Celcius. Typically, use a container as described in example 3. Thereafter, shake the container, and the urine flows out of the capillary. Albumin of the test sample aliquot starts to displace the fluorescein labelled insulin-like peptide of the fluorescein-labelled immuno-complexes of the assay reagent. HCG starts to displace Cy-5-labelled BHCG in the Cy-5-labelled immunocomplexes of the assay reagent.

Immediately after the shaking, place the container in the fluorescence polarisation measurement instrument, having a temperature of 32 degrees of Celsius. For measurement of polarisation of the emitted fluorescence, the mixture in the container is irradiated with polarized light of 475 nm and 650 nm wavelengths and when the polarisation of the emitted light constant, measure its polarisation of emitted light at the wavelengths of 525 nm and 670 nm, with a rather narrow bandwidth, typically 10 nm, if the instruments allows for that. A rather advances dual wavelength irradiation instrument with high precision of polarisation measurements is necessary, because of the overlap of the spectra and the relatively high molecular weight of Cy-5-labelled BHCG. It is preferred to use an instrument which intermittently irradiates at 475 nm and 650 nm, to avoid much spectral interference. Calculate the concentration of human albumin and HCG of the unknown sample by interpolation of the polarisation values of the emitted light at both 525 and 670 nm, measured on a <<standard curve>> obtained by measurement of standards of known human albumin and HCG concentrations. Such a standard curve will be four dimensional since the polarization at both wavelengths will vary with concentrations of both albumin and of HCG. It should be stored stored on the computer of the measurement instrument, enabling a direct calculation of the concentration of albumin and HCG.

If a measurement instrument with a flow cell is used, then bring the mixture to pass through the flow cell for measurement.

If the measurement instrument allows it, much better precision is obtained if—instead of reading the polarisation after the polarisation value has become stable—the polarisation value as a function of time is measured in a kinetic manner. If available, connect the measurement instrument to a computer recording the signal continuously or at different time points, optionally defined by a software. Compare then the recorded values to values obtained by measurement of standards of known human albumin and HCG concentration as a function of time. Optionally, record a data set with polarisation values at the two wavelengths, different times and different human albumin and HCG concentration values, and compare the values obtained with the unknown sample to calculate the human albumin concentration, optionally by the use of the least-square methods for best fit according to standard textbooks of statistics, optionally by means of a computer or another artificial memory connected to the measurement instrument. Furthermore, if you have access to a temperature recording instrument keep the said separate containers (to be combined with unknown blood samples or aliquots of blood samples) at the room temperature, and use the temperature measurement device in or at the fluorescence polarisation instrument, and use an even larger data set with different temperatures included, optionally by the use of the least-square methods for best fit according to standard textbooks of statistics, optionally by means of a computer or another artificial memory connected to the measurement instrument.

The concentration of albumin in urine samples varies significantly. Reference values are in the range of up to 20 mg per liter. Slightly elevated values of uncertain or are found in some healthy individuals, individuals who may stay healthy to old ages. Moderate elevation of the albumin values are seen as early signs of diabetic kidney damage. In severe kidney disease, urine albumin concentration may rise to many hundred mgs per liter. In the performance of this example of the method of the present invention, please therefore adjust the concentration of the fluorescein labelled peptide, and hence the FAB fragment concentration. At high concentration ranges, FAB fragments of high dissociation rate constant must be chosen. At low concentration ranges, use FAB fragments with both high association rate constant and high dissociation rate constant.

Correspondingly, concentration of HCG differ with certain diseases, stage of pregnancy, and obviously with sex. Similar consideration as with the anti-peptide antibody fragment must be used with the anti-BHCG antibodies from which to prepare FAB fragments.

There is an interest to determine both urine HCG and urine albumin in pregnant women.

### Example 18

Simultaneous Measurement of Gonadotropin, Albumin and Immunoglobulin G in Human Urine.

One of the main functions of the kidneys is to excrete urea and other small molecular substances but retain albumin and other proteins in blood (in addition to many other functions). In pregnant women with proteinuria, there is an interest in evaluating the kidneys selectivity between smaller and larger proteins. Simultaneous measurement of albumin and immunoglobulin G is used to assess such selectivity. Normally, the urine contains only trace amounts of albumin and immunoglobulins, but at least 10 timer more albumin than immunoglobulin. With severe impairment of the renal function) such selectively is usually lost.

To obtain a suitable reagent for Simultaneous measurement of gonadotropin, albumin and immunoglobulin G in human urine, use the following protocol:

Use the COSMIX plexing phage display technology referred to above to identify a peptide that binds selectively to Immunoglobulin class G molecules. This is a service that can be bought from the COSMIX GmbH company. Alternatively, digest Protein A into peptide fragments, and identify and purify a IgG binding peptide from the digest, using conventional techniques well known to the art, e.g. as described in Yue et al in The Journal of Biological Chemistry vol 271, p. 22245-22250, 1996. Label the identified and purified peptide with 5-caboxytetramethylrhodamine succidinmindyl ester purchased from Molecular Probes, Inc, using conventional techniques as described in the package insert from aid company. Purify the tetramethylrhodamine-labelled peptide by reversed phase chromatography using conventional techniques described in other examples above.

To the reagent described in example 17, add said tetramethylrhodamine-labelled peptide. If the reagent is intended to measure albumin concentrations e.g. in the concentration range of 50 to 500 mg per liter, add tetramethylrhodamine-labelled peptide to a final concentration of 0.05 micromoles per liter. In this way an impairment of selectivity will be shown as increased binding of IgG to tetramethylrhodamine-peptide.

The final assay reagent is now ready, and optionally add a suitable bacterostatic agent like 0.01% sodium azide for prolonged storage. Keep aliquots of this mixture, e.g. 2 ml, in separate containers to be combined with unknown urine samples or aliquots of urine samples.

At the time for the determination of concentration of albumin, IgG and HCG in a urine samples, take an aliquot, e.g. a volume of 20 ul of the urine sample e.g. by the use of a pre-measured capillary, and combine thereafter the content of this pre-measured capillary with the assay reagent in the said separate container, the said separate container being kept in a place where the temperature is regulated to 32 degrees of Celcius. Typically, use a container as described in example 3. Thereafter, shake the container, and the urine flows out of the capillary. Albumin of the test sample aliquot starts to displace the fluorescein labelled insulin-like peptide of the fluorescein-labelled immuno-complexes of the assay reagent. HCG starts to displace Cy-5-labelled BHCG in the Cy-5-labelled immunocomplexes of the assay reagent. IgG starts to bind to the rhodamine-labelled peptide.

Immediately after the shaking, place the container in the fluorescence polarisation measurement instrument, having a temperature of 32 degrees of Celcius. For measurement of polarisation of the emitted fluorescence, the mixture in the container is irradiated with polarized light of 475 nm, 550 nm and 650 nm wavelengths, using a fluorescence polarization measurement instrument constructed to irradiate the three different wavelengths intermittantly. When the polarisation of the emitted light for each of the different irradiation wavelengths is constant, measure the polarisation of emitted light at the wavelengths of 525 nm, 582 and 670 nm, with a rather narrow bandwidth, typically 10 nm, if the instruments allows for that. A rather advanced irradiation instrument with high precision of polarisation measurements is necessary, because of the overlap of the spectra and the relatively high molecular weight of Cy-5-labelled BHCG. Calculate the concentration of human albumin, IgG and HCG of the unknown sample by interpolation of the polarisation values of the emitted light at both 525, 582 and 670 nm, measured on a <<standard curve>> obtained by measurement of standards of known human albumin, IgG and HCG concentrations. Such a standard curve will be multi dimensional since the polarization at all three, wavelengths will vary with concentrations of both albumin, IgG and of HCG. It should be stored on the computer of the measurement instrument, enabling a direct calculation of the concentration of albumin and HCG.

If a measurement instrument with a flow cell is used, then bring the mixture to pass through the flow cell for measurement.

If the measurement instrument allows it, much better precision is obtained if—instead of reading the polarisation after the polarisation values have become stable—the polarisation values as a function of time is measured in a kinetic manner. If available, connect the measurement instrument to a computer recording the signal continuously or at different time points, optionally defined by a software. Compare then the recorded values to values obtained by measurement of standards of known human albumin, IgG and HCG concentration as a function of time. Optionally, record a data set with polarisation values at the three wavelengths, different times and different human albumin, IgG and HCG concentration values, and compare the values obtained with the unknown sample to calculate the human albumin concentration, optionally by the use of the least-square methods for best fit according to standard textbooks of statistics, optionally by means of a computer or another artificial memory connected to the measurement instrument. Furthermore, if you have access to a temperature recording instrument keep the said separate containers (to be combined with unknown blood samples or aliquots of blood samples) at the room temperature, and use the temperature measurement device in or at the fluorescence polarisation instrument, and use an even larger data set with different temperatures included, optionally by the use of the least-square methods for best fit according to standard textbooks of statistics, optionally by means of a computer or another artificial memory connected to the measurement instrument. In a multi-analyte assay as described, a rather high computing capacity is necessary, but such computing capacity is today easily available, and use soft-ware programs for analysis and calculations of several unknown parameters is preferred, e.g. the use of the Unscrambler prgram, delivered by the CAMO company, Oslo, Norway.

The above examples are all included to explain and clarify the present invention and should not be construed to in any way restrict the protection of the invention as indicated in the enclosed claims.

1. A method for determination of concentration of one or more analytes in a test sample or an aliquot of a test sample of a complex biological fluid, characterized by
a) mixing the said sample or aliquot of the said sample with one single reagent, such as a solid, a solution or premixed solution, wherein said reagent being provided in one side container or compartment of a container, and no other reagent is added during the performance of said method, and said reagent further comprises at least one type of binding molecule with specific affinity for one or more of the said analytes, and said reagent furthermore comprises either fluorescent moieties covalently linked to the said binding molecules or fluorescent analogues of or fluorescent fragments of or fluorescent derivatives of said analyte or analytes, and
b) said mixing resulting in a mixture which is being irradiated with polarized light which permits the exitation of said fluorescent molecules, and
c) measuring the polarisation of the emitted light, and
d) calculating the concentration or concentrations of said analyte or analytes.

2. A method according to claim 1,
characterised by using a reagent for each analyte comprising immunocomplexes between
a) an antibody or an immunoactive fragment of an antibody with specific affinity for said analyte or analytes, and
b) fluorescent analogues or fluorescent fragments of or fluorescent derivatives of said analyte or analytes.

3. A method according to claim 1,
characterised by using a reagent for each analyte comprising complexes between
a) an aptamer or another synthetic binder with a specific affinity for said analyte, and
b) fluorescent analogues or fluorescent fragments of or fluorescent derivatives of said analyte or analytes.

4. A method according to claim 1,
characterised by using a reagent comprising binding molecules with specific affinity for one or more of the said analytes and with fluorescent moieties with absorption maximum between 600 nm and 1000 nm, preferably above 620 nm, covalently linked to the said binding molecules, and said binding molecules being either a peptide or being synthetic binders, optionally being identified by combinatory chemistry techniques or phage display or nucleic acid selection technology.

5. A method according to any of the claims 1 to 4,
characterised by using a reagent comprising fluorescent binding molecules with specific affinity for one analyte, or comprising fluorescent analogues of, or fluorescent fragments of, or fluorescent derivatives of one analyte only.

6. A method according to any of the claims 1 to 5,
characterised by the use of a reagent comprising different fluorescent moieties covalently bound to different binding molecules with different specific affinities.

7. A method according to any of the claims 1 to 6,
characterized by the use of a reagent comprising one or more peptides or derivatives of peptides with specific binding affinity for an analyte, said binding peptides having a fluorescent residue covalently linked and being constituted by less than 30 amino acids.

8. A method according to claim 7,
characterised in that binding peptide is constituted by less than 20 amino acids.

9. A method according to claim 7,
characterised in that binding peptide is constituted by less than 15 amino acids.

10. A method according to any of the claims 1 to 9,
characterised by the use of a reagent comprising peptids or derivatives of peptids containing amino acid sequence Ala-Arg-Asn-Arg-Asn or Ala-Arg-Asn-Gly-Asn for quantitation of C-reactive protein.

11. A method according to any of the claims 1 to 10,
characterised by the use of a reagent with fluorescent residues with maximum coefficient of absorption at a wavelength above 640 nm.

12. A method according to any of the claims 1 to 11,
characterised by the use of a reagent comprising cell lysing substances or anti-coagulants or detergents.

13. A method according to any of the claims 1 to 12,
characterised by the use of a reagent comprising one or more fluorescent moieties selected from the group consisting of fluoresceine, Texas Red, Cy5, other Cy Dye FluorLink substances, other Cyanin derivatives, Rhodamin, Methyl Rhodamin, Biodypi 630/650-X/MeOH; Biodypi 650/655-X/MeOH, Biodypi FL/MeOH Biodypi R6G/MeOH, Biodypi TMR-X/MeOH Biodypi TR-X/MeOH or other substances from the Biodipy group of substances, Alexa Fluor Dyes of different wavelengths, Ruthenium ligand complexes, lanthanoid elements such as Europium, Samarium or Terbium complex bound to a chelating ligand like DTPA, EDTA or NI.

14. A method according to any of the claims 1 to 13,
characterised by that the polarisation of the emitted light is measured as a function of time, either as a continuous kinetic reading or a reading of the change in polarisation of the emitted light between two or more time points, or as a measurement of the polarisation of the omitted light after a defined point of time.

15. A method according to any of the claims 1 to 14,
characterised by that sample material or aliquot of the sample material is constituted by a biological material, or a dilution or an extract or being dissolved from or being filtrated from the said biological material.

16. A method according to any of the claims 1 to 15,
characterised by that sample material or aliquot of the sample material is constituted by blood, or blood serum, or blood plasma, or blood cells, or lysate from blood or blood calls, or urine, or cerebrospinal fluid, or tear liquid, or sputum, or semen, or plasma, or semen or material aspirated from the gastro-intestinal tract or feces, or extract or filtrate of suspension of feces, or plant material or extracts thereof, or dissolved plant material or filtrate thereof.

17. A method according to any of the claims 1 to 16,
characterised by the use of standards or calibrators comprising known concentrations of the analyte or the analytes, and furthermore wherein the concentration or concentrations of said analyte or analytes in unknown sampler is calculated by interpolation of the values obtained from the unknown samples on the standard curve obtained from said known standards or calibrators.

18. A method according to any of the claims 1 to 17,
characterised by the use of a standard curve stored in an artificial memory, optionally connected to the fluorescent polarisation instrument in use.

19. A method according to any of the claims 1 to 18,
characterized by the use of temperature correction algorithms, either generated empirically or theoretically, to compensate for differences in fluorescence polarisation caused by differences in temperature at different time of measurements of standards and unknown samples, or between standards, or between unknown samples.

20. A method according to any of the claims 1 to 19,
characterised by being provided in concentrated or dry form, to be diluted or reconstituted before use, the said reagent being provided divided between different compartments for combination into one reagent prior to use.

21. A reagent for the performance of the method according to any of the claims 1 to 20,
characterised in that said reagent comprises at least one type of binding molecule with specific affinity for one or more of the said analytes, and said reagent furthermore comprises fluorescent moities covalently linked to the said binding molecules or fluorescent analogues of or fluorescent fragments of or fluorescent derivatives of said analyte or analytes.

22. A reagent according to claim 21,
characterised in that the reagent comprises complexes between
a) an antibody or an immunoactive fragment of an antibody or an aptamer or a synthetic binder with specific affinity for at least one analyte and
b) fluorescent analogues or fluorescent fragments of or fluorescent derivatives of said analyte or analytes.

23. A reagent according to claims 21 to 22,
characterised in comprising binding molecules with specific affinity for one or more of the said analyte and optionally with fluorescent moities with absorption maximum between 600 nm and 1000 nm, preferably exceeding 620 nm, more preferably exceeding 640 nm, covalently linked to the said binding molecules, and said binding molecules being either of peptide or aptamer composition or being synthetic binders, optionally being identified by combinatory chemistry techniques or phage display or nucleic acid selection technology.

24. A reagent according to claims 21 to 23,
characterised in being an assay reagent comprising peptid binders or binders of derivatives of peptids, including fluorescent derivatives of said binders, containing the amino acid sequence Ala-Arg-Asn-Arg-Asn and/or Ala-Arg-Asn-Gly-Asn.

25. Use of the method according to claims 1 to 20 to determine concentrations of clinically related substances in samples of biological material from living organisms in need thereof.

26. Kit for the determination of concentration of one or more analytes in a test sample or an aliquot of a test sample of complex biological fluid,
characterized in comprising one or more containers, wherein the container(s) or compartment of the container(s) contains one single reagent, preferably in the fluidal state and according to any of the claims 21-24, and wherein the reagent comprises one or more fluorescensce-labelled specific binding molecules towards the analyte(s) to be measured, or a fluorescence-labelled analogue or a fluorescent fragment or a fluorescent derivative of said analyte(s), as well as device for obtaining the exact volume(s) of the complex biological fluid to be tested and that is needed in order to perform the method adequately.

27. Kit according to claim 26,
characterized in that the reagent which is contained in a container or a compartment of a container, is formed to a ready-for-use reagent by mixing the content from different containers prior to or immediately prior to or in connection with the execution of the analysis.
